SEQUENCE LISTING (3 sequences)

SEQ ID NO: 1 - protein, 500 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRALAPRGPARAEGAQDAGHHHGRLHALLAALLPGQRGEGLPPRAGARPPLRLLQLAGLRQLGLQPHHLLPQPRLPQGLPATALLRAQGCPPAPRDPRRPAARLGLSGPARTPAIARGRLGRRRRRCRRGHAARAPAGALGRLQRRGGGGQRLEPGRAVPPRLRLGIQGVGPGAGRGLRARLPRGTRRSVFT

SEQ ID NO: 2 - protein, 26 aa, Homo sapiens
HWWRAESDEARRCYNDPKCCDFVTNR

SEQ ID NO: 3 - DNA, 1503 bp, Homo sapiens
atgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcgggccctcgcgcctcgtggccctgcgcgagcagaaggcgctcaggacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagcgactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtgtagggcccggcgcggggcgcggactccgggcacggcttcccaggggaacgaggagatctgtgtttacttaa

(19) United States
(12) Patent Application Publication
(10) Pub. No.: US 2024/0124594 A1
(43) Pub. Date: Apr. 18, 2024

(54) TREATMENT OF CVD AND SYSTEMIC SCEROSIS WITH BETA-1 ADRENERGIC RECEPTOR ANTIBODIES

(71) Applicant: The Cleveland Clinic Foundation, Cleveland, OH (US)

(72) Inventors: W.H. Wilson Tang, Cleveland, OH (US); Sathyamangla V. Naga Prasad, Cleveland, OH (US); Maradumane Mohan, Cleveland, OH (US)

(21) Appl. No.: 18/263,929

(22) PCT Filed: Feb. 2, 2022
(86) PCT No.: PCT/US22/14951

§ 371 Date: Aug. 2, 2023

Related U.S. Application Data
(60) Provisional application No. 63/144,735, filed on Feb. 2, 2021.

(51) Int. Cl. C07K 16/28; A61K 31/138; A61P 9/04; C07K 14/00; C12N 15/86

(52) U.S. Cl. CPC C07K 16/286; A61K 31/138; A61P 9/04; C07K 14/001; C12N 15/86

(57) ABSTRACT

The present invention relates to systems, kits, and methods for treating a subject that has cardiovascular disease or Systemic Sclerosis with at least one of the following: a) IgG3 β1-Adrenergic Receptor antibodies (IgG3 β1AR antibodies), or antigen-binding portion thereof; b) an antigenic protein that elicits the production of IgG3 β1AR antibodies in the subject; or c) a vector comprising a nucleic acid sequence encoding the antigenic protein or IgG3 β1AR antibodies. In certain embodiments, the subject has dilated cardiomyopathy. In some embodiments, the subject is also administered a beta-blocker.

The present application claims priority to U.S. Provisional application Ser. No. 63/144,735, filed Feb. 2, 2021, which is herein incorporated by reference in its entirety.

### STATEMENT REGARDING FEDERAL FUNDING

This invention was made with government support under grant number HL089473 awarded by the National Institutes of Health. The government has certain rights in the invention.

### FIELD OF THE INVENTION

The present invention relates to systems, kits, and methods for treating a subject that has cardiovascular disease or Systemic Sclerosis with at least one of the following: a) IgG3 β1-Adrenergic Receptor antibodies (IgG3 β1AR antibodies), or antigen-binding portion thereof; b) an antigenic protein that elicits the production of IgG3 β1AR antibodies in the subject; or c) a vector comprising a nucleic acid sequence encoding the antigenic protein or IgG3 β1AR antibodies. In certain embodiments, the subject has dilated cardiomyopathy. In some embodiments, the subject is also administered a beta-blocker.

### BACKGROUND

β-adrenergic receptors (βARs) are one of the most well studied proto-typical 7-trans-membrane receptors that are powerful regulators of cardiac function (Rockman et al., 2002; Vasudevan et al., 2011a). Among the βARs, β1- and β2-ARs are highly expressed in the myocardium (Rockman et al., 2002). Catecholamine binding to the βARs results in G-protein coupling leading to adenylyl cyclase activation, mediating cAMP-protein kinase A (PKA) signal cascade (Wallukat, 2002). Activation of cAMP-PKA cascade alters calcium cycling resulting in increased myocardial contractility. Although β3- and β2-ARs play a key role in myocardial contraction, there is also increasing appreciation that beyond contraction, they may have distinct roles to play in phenotypic outcomes (Dungen et al., 2020; Liaudet et al., 2014). Increasing evidence from in vivo and in vitro studies show functional divergence between β1AR and β2ARs. Cellular studies show that chronic catecholamine stimulation leads to cardiomyocyte apoptosis through activation of β1ARs, while β2ARs may mediate cardioprotective signaling (Milano et al., 1994; Steinberg, 2018). Consistently, cardiomyocyte overexpression of β1ARs leads to maladaptive cardiac remodeling, while overexpression of β2ARs results in hypertrophic response, reflecting their divergent roles (Engelhardt et al., 1999; Steinberg, 2018). However, β1AR downregulation (loss of cell surface receptors) and desensitization (inability to be activated by catecholamine) are one of the key hallmark features of human heart failure (Port and Bristow, 2001; Steinberg, 2018).

Dilated cardiomyopathy (DCM) is one the most commonly observed phenotype of heart failure associated with progressive loss in ventricular function, wherein idiopathic DCM represents pathogenesis without a specific known cause (Magnusson et al., 1994; Wynne, 1988). Patients with idiopathic DCM have a diagnosis of diverse etiologies and varied presentations however, dysregulation of the immune system is considered to be one of the central players in cardiac pathogenesis. The human immune system is a complex multicellular regulated defense mechanism that is characterized by inter-individual variability in response to a similar stress/injury. In healthy homeostatic conditions, it is designed to discriminate between self and foreign components, and clear components deemed to be foreign (Crampton et al., 2010; Kaya et al., 2012; Mann, 2011). However, when this regulatory control is lost, it leads to pathological circumstances wherein self-components are attacked, resulting in auto-immune disease (Crampton et al., 2010; Kaya et al., 2012; Mann, 2011). Thus, autoantibodies generated against the self-antigens exacerbate and may accelerate the disease progression (Crampton et al., 2010; Kaya et al., 2012; Mann, 2011).

Circulating autoantibodies have been identified in heart failure and increasing evidence suggests that they may be critically linked to heart failure pathogenesis. Autoantibodies against β1AR have been observed in patients with heart failure (30-40%), and are positively co-related with the phenotype (Baba, 2010; Iwata et al., 2001; Jahns et al., 1999b; Magnusson et al., 1990; Magnusson et al., 1994; Nagatomo et al., 2009; Stork et al., 2006). Studies have shown that β1AR autoantibody stabilizes the receptor in an active form prolonging its activation mimicking catecholamines (Deubner et al., 2010). Furthermore, β1AR autoantibodies can elevate the L-type Ca²⁺ current increasing in vitro contractility (Christ et al., 2001). This elevated and prolonged activation reflects the hyper-sympathetic state associated with deleterious cardiac remodeling and DCM. Although β-blockers ameliorate the signaling from the sympathetic overdrive, their role in upregulation of βARs is thought to underlie the worsening outcomes of heart failure due to binding by β1AR autoantibodies.

### SUMMARY OF THE INVENTION

The present invention relates to systems, kits, and methods for treating a subject that has cardiovascular disease or Systemic Sclerosis with at least one of the following: a) IgG3 β1-Adrenergic Receptor antibodies (IgG3 β1AR antibodies), or antigen-binding portion thereof; b) an antigenic protein that elicits the production of IgG3 β1AR antibodies in the subject; or c) a vector comprising a nucleic acid sequence encoding the antigenic protein or IgG3 β1AR antibodies. In certain embodiments, the subject has dilated cardiomyopathy. In some embodiments, the subject is also administered a beta-blocker.

In some embodiments, provided herein are methods of treating a subject that has cardiovascular disease (CVD), or Systemic Sclerosis (SS), comprising: treating a subject that has CVD or SS with at least one of the following: a) IgG3 β1-Adrenergic Receptor antibodies (IgG3 β1AR antibodies) or antigen-binding portion thereof (e.g., either of which are derived from the subject themselves); b) an antigenic protein that elicits the production of the IgG3 β1AR antibodies in the subject; or c) a vector comprising a nucleic acid sequence encoding: i) the IgG3 β1AR antibodies, ii) antigen-binding portion thereof, or iii) the antigenic protein.

In certain embodiments, provided herein are kits, system, or compositions comprising: a) at least one of the following: i) IgG3 β1-Adrenergic Receptor antibodies (IgG3 β1AR antibodies) or antigen-binding portion thereof; ii) an antigenic protein that elicits the production of IgG3 β1AR antibodies in a subject; or iii) a vector comprising a nucleic acid sequence encoding: i) the IgG3 β1AR antibodies, ii) the antigen-binding portion thereof, or iii) the antigenic protein, and b) a β-blocker.

In further embodiments, provided herein are methods comprising: a) receiving results of, or conducting, an IgG3 β1-Adrenergic Receptor IgG3 β1AR antibodies (IgG3 β1AR antibodies) level analysis on a sample from a subject with cardiovascular disease (CVD) or Systemic Sclerosis, and b) performing at least one of the following after identifying the sample as having higher levels of the IgG3 β1AR antibodies compared to control levels, i) treating the subject with a beta-blocker; ii) treating the subject with IgG3 β1AR antibodies or antigen-binding portion thereof; iii) treating the subject with an antigenic protein that elicits the production of the IgG3 β1AR antibodies in the subject; iv) a vector comprising a nucleic acid sequence encoding: i) the IgG3 β1AR antibodies, ii) the antigen-binding portion thereof, or iii) the antigenic protein; and/or v) providing a report to the patient or medical personnel treating the patient, indicating the subject is suitable for, or should be, treated with: A) the beta-blocker, B) the IgG3 β1AR antibodies or antigen-binding portion thereof, C) the antigenic protein, and/or D) the vector. In particular embodiments, the control levels are from the general population or CVD patients not stratified by IgG3 β1AR antibody levels.

In certain embodiments, the subject is negative or lower than control (e.g., general population of CVD or systemic sclerosis patients) for IgG3 β1AR antibodies prior to the treatment. In other embodiments, the CVD comprises dilated cardiomyopathy. In further embodiments, the IgG3 β1AR antibodies are monoclonal. In some embodiments, the methods further comprise: testing a blood, serum, or plasma sample from the subject for the presence of IgG3 β1AR antibodies prior to the treatment. In other embodiments, the methods further comprise: testing a blood, serum, or plasma sample from the subject for the presence of IgG3 β1AR antibodies after the treatment.

In particular embodiments, the antigenic protein comprises at least a portion of SEQ ID NO:1 (e.g., 25% . . . 50% . . . 75% . . . or 100% of SEQ ID NO:1). In other embodiments, the antigenic protein comprises SEQ ID NO:2. In further embodiments, the antigenic protein comprises at least 15 consecutive amino acids from SEQ ID NO:2. In certain embodiments, the antigenic protein comprises a peptide with at least 90% amino acid sequence identity with SEQ ID NO:2. In additional embodiments, the nucleic acid sequence comprises at least 50 consecutive nucleotides from SEQ ID NO:3.

In some embodiments, the subject is a human. In further embodiments, the IgG3 β1AR antibodies, or antigen-binding portion thereof, are human or humanized antibodies, and the subject is a human. In particular embodiments, the treating comprises intravenous treatment. In other embodiments, the vectors comprises a plasmid, adeno-associated virus, or adeno-associated virus.

In some embodiments, the subject is on a β-blocker prior to the treatment. In other embodiments, the methods further comprise: administering a β-blocker to the subject prior to, during, or after the treatment. In certain embodiments, the β-blocker is selected from the group consisting of Acebutolol, Atenolol, Bisoprolol, Metoprolol, Nadolol, Nebivolol, and Propranolol. In certain embodiments, the IgG3 β1AR antibodies are autoantibodies purified from a blood, serum, or plasma sample of a donor or the subject themselves.

### BRIEF DESCRIPTION OF THE FIGURES

FIG. 1. Generation and characterization of stable Human Embryonic Kidney (HEK) cell line expressing human β1-Adrenergic Receptors (β1AR). (A) Parental HEK293 cells and HEK293 cells overexpressing FLAG-human-β1AR (HEK-β1AR) were lysed with NP-40 lysis buffer, cell lysates (50 μg each) were subjected to SDS-PAGE and immunoblotted with anti-FLAG antibody. The blots were stripped and immunoblotted with anti-β-actin antibody as loading control. (B) Isolated plasma membranes from HEK-β1AR cells were used to perform the receptor binding assay using ¹²⁵I-Cyanopindolol to show the expression of receptors on the cell membranes and binding curve generated establish very high expression of the receptors (n=3). (C) HEK-β1AR cells were serum starved for 4 h and stimulated with 10 μM Dobutamine (Dob) or 10 μM Metoprolol (Meto) for 10 min. The cells were lysed and cyclic Adenosine Mono Phosphate (cAMP) generation was measured using cAMP assay kit. Bar graphs represent cumulative data (n=3 independent experiments and each experiment is performed in triplicates). *p≤0.05 Ctrl vs Dob, **p≤0.01 Meto vs Dob/Ctrl. (D) Isolated membranes from HEK-β1AR cells were used to perform Adenylate Cyclase (AC) assay in vitro to assess the function of the expressed receptors in the presence of Dob or Meto. The amount of cAMP generated by control is expressed as 100 percent and percent change in generation of cAMP in treatments are shown (n=3). *p<0.05 Ctrl vs Dob, **p<0.001 NaF vs Ctrl/Dob/Meto. (E) HEK-β1AR cells were serum starved and stimulated with Dob or Meto. The cell lysates were subjected to western immunoblotting with anti-phospho-ERK. The blots were stripped and immunoblotted with anti-ERK antibody as loading control (left panel). (F) Cumulative densitometric data are presented as bar graphs (n=3). *p≤0.0001, Dob vs Ctrl/Meto (right panel).

FIG. 2. Autoantibodies (AAb) positive human serum alter β1AR function. (A) HEK-β1AR cells were serum starved treated with human serum negative for IgG3 class of AAb [IgG3(−)] or positive for IgG3 class of AAb [IgG3(+)] for 10 min. The cAMP was measured (n=3 to 4 independent patient serum samples). *p<0.01 IgG3(−) vs Ctrl/IgG3(+). (B) HEK-β1AR cells were serum starved, pre-treated with IgG3(−) human serum for 30 min and stimulated with Dob or Meto. The cAMP was measured (n=3 independent patients). *p<0.05 Meto vs Ctrl. (C) HEK-β1AR cells were serum starved, pre-treated with IgG3(+) human serum and stimulated with Dob or Meto. The cAMP was measured (n=4). *p<0.05 Meto vs Ctrl/Dob. (D) Isolated membranes from HEK-β1AR cells were pre-treated with IgG3(−) human serum, or IgG3(+) human serum, AC activity was performed in the presence of Dob or Meto. The amount of cAMP generated by control is expressed as 100%. % change in generation of cAMP following treatments were calculated and Δ-change compared to control is represented (n=4 (no serum), 5 (IgG3(−) serum) or 7 (IgG3(+) serum)). **p<0.05 Dob human serum vs Ctrl human serum. *p<0.01 Meto human serum vs Dob human serum.

FIG. 3. Autoantibodies (AAb) alter β1AR function. (A) HEK-β1AR cells were serum starved, pre-treated with affinity purified IgG3(−) AAb for 30 min and stimulated with Dob or Meto. The cAMP was measured (n=9). Δ-change compared to control is represented. *p<0.05 Meto vs Dob [IgG3(−)]. **p<0.05 Meto vs Dob [IgG3(+)]. $p<0.01 Meto [IgG3(+)] vs Meto [IgG3(−)]. (B) Isolated membranes from HEK-β1AR cells were pre-treated with IgG3(−) purified IgG, or IgG3(+) purified IgG, AC activity was performed in the presence of Dob or Meto. The amount of cAMP generated by vehicle treatment (control) is expressed as 100%. % change in generation of cAMP following treatments were calculated and Δ-change compared to control is represented (n=4 (no autoantibody, 5 (purified IgG3(−) autoantibody) or 7 (purified IgG3(+) autoantibody)). **p<0.05 Meto IgG3(+) purified IgG vs Dob IgG3(+) purified IgG.

FIG. 4. Autoantibodies (AAb) alter β-arrestin recruitment. (A) HTLA cells (HEK cells stably expressing tTA-dependent luciferase reporter and a β-arrestin2-TEV fusion gene) were transfected with ADBR1-Tango (β1AR) gene and pre-treated with human sera negative for IgG3 autoantibodies or positive for IgG3 autoantibodies for 30 min, subjected to β-arrestin recruitment assay and compared with No IgG control (n=8). *p<0.001 Dob IgG3(+) serum vs Dob IgG3(−) serum and Dob No IgG. (B) HTLA cells (HEK cells expressing tTA-dependent luciferase reporter and a β-arrestin2-TEV fusion gene) were transfected with ADBR1-Tango (β1AR) gene and pre-treated with affinity purified IgG3(−) or IgG3(+) autoantibodies for 30 min, subjected to β-arrestin recruitment assay and compared with No IgG control (n=8). *p<0.001 Dob IgG3(+) autoantibodies vs Dob IgG3(−) autoantibodies or Dob No IgG.

FIG. 5. Autoantibodies (AAb) alter membrane localization of β-arrestin. (A) HEK-β1AR cells transfected with plasmids containing β-arrestin2-GFP fusion gene were plated on poly-L-Lysine coated coverslips, serum starved, pre-treated with No IgG, affinity purified IgG3(−) or IgG3(+) autoanitibody, stimulated with Dob, fixed with 4% paraformaldehyde for 30 min and mounted using prolong gold mountant with DAPI. The β-arrestin recruitment was assessed using confocal microscopy. Representative images of β-arrestin recruitment depicted by green fluorescence (bar=10 μm). (B) A representative depiction of plot profile measurement of GFP fluorescence showing changes in fluorescence intensities from the one edge to the other edge of the cell which depicts the reduction of fluorescene intensity in the cytoplasm and increase at the plasma membrane following Dob treatment. (C) The relative ratio of plasma membrane (Mem) to cytoplasm (Cyt) intensity with Dob treatment in presence of No IgG, IgG3(−) or IgG3(+) autoantibodies (n=3). *p<0.05 IgG3(+) vs. IgG3(−) autoantibodies or No IgG. (D) Average changes (i.e. clearance/loss) in cytoplasmic fluorescence intensity calculated over total fluorescence intensity of the cell reflecting 3-arrestin recruitment, represented as percentage [˜30 cells/experiment (n=3)]. *p<0.05 IgG3(+) autoantibodies vs No IgG or IgG3(−) autoantibodies. (E) Percentage of cells showing effective β-arrestin recruitment following Dob in presence of No IgG, IgG3(−) or IgG3(+) autoantibodies [˜30 cells/experiment) (n=3)]. *p<0.05 IgG3(+) autoantibodies vs No IgG or IgG3(−) autoantibodies.

FIG. 6. Autoantibodies (AAb) alter β1AR signaling. (A) HEK-β1AR cells were serum starved for 4 h, pre-treated with IgG3(−) or IgG3(+) human serum for 30 min and stimulated with Dob or Meto. The cell lysates were subjected to western immunoblotting with anti-phospho-ERK antibody. The blots were stripped and immunoblotted with anti-ERK antibody as loading control. (B) Cumulative data for cells pre-treated with IgG3(−) human serum (n=3). (C) Cumulative data for cells pre-treated with IgG3(+) human serum (n=3). (D) HEK-β1AR cells were serum starved, pre-treated with No IgG, affinity purified IgG3(−) or IgG3(+) AAb and stimulated with Dob or Meto. The cell lysates were subjected to western immunoblotting as above. (E) Cumulative data (n=3). *p<0.05 Ctrl IgG3(−)/IgG3(+) vs Ctrl No IgG. **p<0.01 Dob IgG3(−)/IgG3(+) vs Dob No IgG. $p<0.05 Meto IgG3(+) vs Meto No IgG/IgG3(−).

FIG. 7. Autoantibodies (AAb) alter β1AR function in human hearts. (A) Isolated membranes from donor human heart tissues were pre-treated with no autoantibody, purified IgG3(−) autoantibody or purified IgG3(+) purified autoantibody and AC activity was performed in the presence of Dob or Meto. The amount of cAMP generated by control is expressed as 100%, % change in generation of cAMP following treatments were calculated and Δ-change compared to control is represented (n=4). *p<0.05 Dob IgG3(+) purified IgG vs Dob IgG(−) purified IgG/No IgG. **p<0.005 Meto IgG3(+) purified IgG vs Meto IgG3(−) purified IgG/No IgG. (B) AC activity performed as above. The Dob mediated cAMP is expressed as percent over control. *p<0.05 IgG3(−) purified IgG vs No IgG. **p<0.05 IgG3(+) purified IgG vs IgG3(−) purified IgG/No IgG. (C) AC activity performed as above. The Meto mediated cAMP is expressed as percent over control. *p<0.05 IgG3(+) purified IgG vs IgG3(−) purified IgG/No IgG.

FIG. 8. Autoantibodies (AAb) positive human serum does not alter β2AR signaling. (A) HEK293 cells overexpressing FLAG-human-β2AR (HEK-β2AR) were serum starved for 4 h, pre-treated with no IgG, IgG3(−) or IgG3(+) human serum for 30 min and stimulated with 10 μM Isoproterenol (Iso) or 10 μM ICI (ICI) for 10 min lysed with NP-40 lysis buffer, cell lysates (50 μg each) were subjected to SDS-PAGE and immunoblotted with anti-phospho-β2AR antibody. The blots were stripped and immunoblotted with anti-FLAG antibody as loading control. The cell lysates were subjected to western immunoblotting with anti-phospho-β2AR. The blots were stripped and immunoblotted with anti-FLAG antibody as loading control. (B) Cumulative data for cells pre-treated with no IgG3 human serum (n=3). *p<0.01 Iso vs Ctrl/ICI (C) HEK-β2AR cells were serum starved, pre-treated with IgG3(−) human serum and stimulated with Iso or ICI. The cell lysates were subjected to western immunoblotting as above (n=3). *p<0.01 Iso vs Ctrl/ICI. (D) HEK-β2AR cells were serum starved, pre-treated with IgG3(+) human serum and stimulated with Iso or ICI. The cell lysates were subjected to western immunoblotting as above (n=4). *p<0.01 Iso vs Ctrl/ICI. (E) HEK-β2AR cells were serum starved, pre-treated with no IgG, IgG3(−) or IgG3(+) human serum and stimulated with Iso and ICI. The cell lysates were subjected to western immunoblotting with anti-phospho-ERK. The blots were stripped and immunoblotted with anti-ERK antibody as loading control. (F) Cumulative data for cells pre-treated with no IgG human serum (n=3). *p<0.01 Iso vs Ctrl/ICI. (G) Cumulative data for cells pre-treated with IgG3(−) human serum (n=3). *p<0.05 Iso vs Ctrl/ICI. (H) Cumulative data for cells pre-treated with IgG3(+) human serum (n=4). *p<0.05 Iso vs Ctrl/ICI.

FIG. 9. Illustration depicting signaling mechanism of IgG3(+) autoantibodies generated against human β1AR. (A) Agonist mediated β1AR signaling. (B) Antagonist mediated β1AR signaling. (C) Agonist mediated β1AR signaling modulated by IgG3(+) autoantibodies. (D) Antagonist mediated β1AR signaling modulated by IgG3(+) autoantibodies.

FIG. 10. Autoantibodies (AAb) alter β1AR function. (A) Isolated membranes from HEK-β1AR cells were pre-treated with IgG3(−) human serum and AC activity was performed in the presence of Dob or Meto. The amount of cAMP generated by control is expressed as 100 percent and percent change in generation of cAMP in treatments are shown (n=4, 5). *p≤0.05 Meto human serum vs Ctrl/Dob human serum. (B) Isolated membranes from HEK-β1AR cells were pre-treated with IgG3(+) human serum and AC activity was performed in the presence of Dob and Meto. cAMP generated are shown as above (n=4, 7). *p<0.05 Dob human serum vs Ctrl/Meto human serum. **p<0.01 Meto human serum vs Ctrl human serum.

FIG. 11. Autoantibodies (AAb) alter β1AR function. (A) HEK-β1AR cells were serum starved treated with affinity purified IgG3(−) and IgG3(+) AAb for 10 min. The cAMP was measured (n=3, 9). (B) HEK-β1AR cells were serum starved, pre-treated with affinity purified IgG3(−) AAb and stimulated with Dob or Meto. The cAMP was measured (n=9). *p≤0.05 Meto vs Dob. (C) HEK-β1AR cells were serum starved, pre-treated with affinity purified IgG3(+) Aab and stimulated with Dob or Meto. The cAMP was measured (n=9). *p≤0.01 Meto vs Ctrl/Dob. (D) Isolated membranes from HEK-β1AR cells were pre-treated with IgG3(−) purified IgG, AC activity was performed in the presence of Dob or Meto. The amount of cAMP generated by control is expressed as 100 percent and percent change in generation of cAMP in treatments are shown (n=4, 5). **p<0.05 Meto vs Ctrl IgG3(−). (E) Isolated membranes from HEK-β1AR cells were pre-treated with IgG3(+) purified IgG, AC activity was performed in the presence of Dob and Meto. cAMP generated are shown as above (n=4, 7). *p<0.05 Dob vs Ctrl. **p<0.001 Meto vs Dob.

FIG. 12. Autoantibodies (AAb) alter β1AR signaling. (A) HEK-β1AR cells were serum starved, pre-treated with affinity purified IgG3(−) or IgG3(+) AAb and stimulated with Dob or Meto. The cell lysates were subjected to western immunoblotting for anti-phospho-ERK antibody. The blots were stripped and immunoblotted with anti-ERK antibody as loading control. (B) Cumulative data for cells pre-treated with affinity purified IgG3(−) AAb (n=5). *p<0.0001 Dob vs Ctrl/Meto. (C) Cumulative data for cells pre-treated with affinity purified IgG3(+) Aab (n=6, 7). *p<0.0001 Dob vs Ctrl/Meto.

FIG. 13. Effect of Agonist and Inverse Agonist on β2AR signaling. (A) HEK-β2AR cells were serum starved and stimulated with Iso or ICI. The cell lysates were subjected to western immunoblotting for anti-phospho-β2AR antibody. The blots were stripped and immunoblotted with anti-FLAG antibody as loading control. (B) Cumulative densitometric data for the blots (n=3). *p<0.05 Iso vs Ctrl/ICI. (C) HEK-β2AR cells were serum starved and stimulated with Iso and ICI, lysed and immunoblotted with anti-phospho-ERK antibody. The blots were stripped and immunoblotted with anti-ERK antibody as loading control. (D) Cumulative densitometric data for the blots (n=3). *p<0.05 Iso vs Ctrl/ICI.

FIG. 14. A) Shows the amino acid sequence of Human Beta-1 Adrenergic Receptor (SEQ ID NO: 1). B) Shows the mRNA sequence of Human Beta-1 Adrenergic Receptor (SEQ ID NO:3).

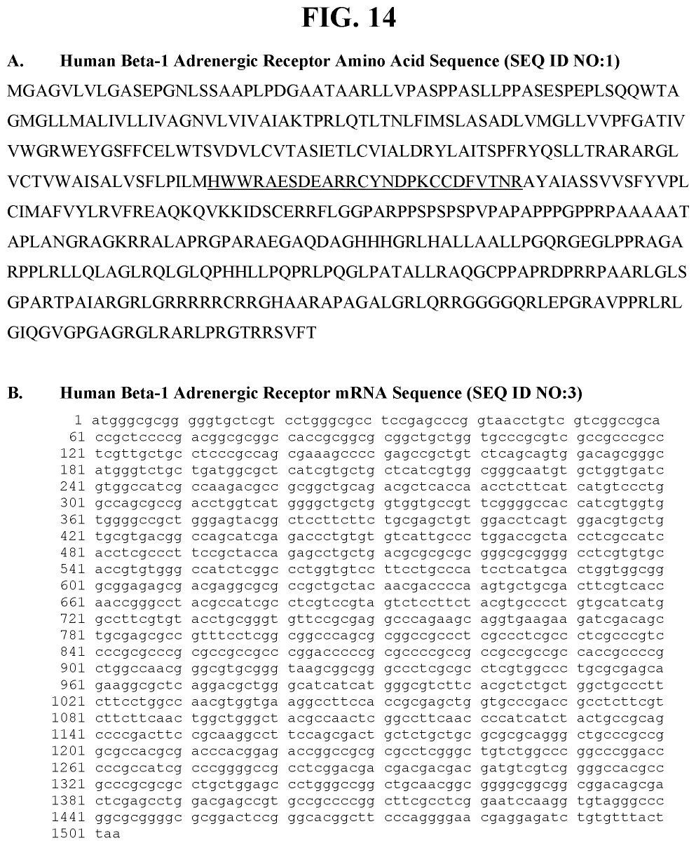

FIG. 15. Shows the format for the enzyme-linked immunosorbent assays (ELISAs) performed in Example 2 to investigate the presence of circulating IgG and IgG3 subtype β1AR-AAbs.

FIG. 16. Shows the results from Example 2 where it was found, of the total SSc cohort, 11 (31%) and 7 (19%) were positive for anti-β1AR IgG and IgG3 respectively; between SSc patients with and without cardiac involvement, there was no significant difference in IgG or IgG3 positivity; and there was a significant increase in IgG3 positivity in the lcSSc group compared to the dcSSc plus ssSSc group at the 2× control threshold (p=0.011) (FIG. 16).

### DEFINITIONS

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, dilated cardiomyopathy, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

The phrase “dilated cardiomyopathy” refers to a condition in which the heart becomes enlarged and cannot pump blood effectively. Symptoms vary from none to feeling tired, leg swelling, and shortness of breath. It may also result in chest pain or fainting. Complications can include heart failure, heart valve disease, or an irregular heartbeat. Causes include genetics, alcohol, cocaine, certain toxins, complications of pregnancy, and certain infections. Coronary artery disease and high blood pressure may play a role, but are not the primary cause. In many cases the cause remains unclear.

As used herein “heart failure” refers to when the heart is unable to pump sufficiently to maintain blood flow to meet the body's needs. Signs and symptoms of heart failure commonly include shortness of breath, excessive tiredness, and leg swelling. The shortness of breath is usually worse with exercise, while lying down, and may wake the person at night. A limited ability to exercise is also a common feature. Common causes of heart failure include coronary artery disease including a previous or current myocardial infarction (heart attack), high blood pressure, atrial fibrillation, valvular heart disease, excess alcohol use, infection, and cardiomyopathy of an unknown cause.

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is specifically a human subject (e.g., with dilated cardiomyopathy).

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

### DETAILED DESCRIPTION OF THE INVENTION

The present invention relates to systems, kits, and methods for treating a subject that has cardiovascular disease or Systemic Sclerosis with at least one of the following: a) IgG3 β1-Adrenergic Receptor antibodies (IgG3 β1AR antibodies), or antigen-binding portion thereof (e.g., derived from the subject themselves or other donor); b) an antigenic protein that elicits the production of IgG3 β1AR antibodies in the subject; or c) a vector comprising a nucleic acid sequence encoding the antigenic protein or IgG3 β1AR antibodies. In certain embodiments, the subject has dilated cardiomyopathy. In some embodiments, the subject is also administered a beta-blocker.

β1AR autoantibodies belongs to IgG class of immunoglobulins that can be further sub-classified into IgG 1, 2, 3 and 4 (Schur, 1988; Vidarsson et al., 2014). In work conducted during development of embodiments herein, serum from patients containing IgG3-positive [IgG3(+)] or -negative [IgG3(−)], or purified β1AR autoantibodies were used in cellular studies. HEK 293 cells stably expressing human β1AR were pre-treated with β1AR autoantibodies followed by β1AR-selective agonist (dobutamine) or antagonist/blocker (metoprolol). Given the specificity of the auto-antibody towards human β1ARs, we used non-failing human heart samples as an endogenous system to assess adenylyl cyclase activity in the presence of β1AR autoantibodies. This work showed that the IgG3 subclass of β1AR autoantibodies markedly attenuates agonist mediated G-protein signaling by promoting enhanced β-arrestin binding, while preserving G-protein independent ERK activation. Uniquely, β1AR autoantibodies biases β-blocker signal towards G-protein coupling, unraveling a distinctive signaling role for the IgG3 sub-class of β1AR autoantibodies.

IgG3 β1-Adrenergic Receptor antibodies (IgG3 β1AR antibodies) (and antigen-binding portions thereof) may be prepared using various immunogens. In some embodiments, all or a portion of SEQ ID NO:1 (e.g., the 26 amino acid SEQ ID NO:2) may be employed to generate antibodies that recognize β1AR. Such antibodies include, but are not limited to polyclonal, monoclonal, chimeric, single chain, Fab fragments, and Fab expression libraries. Antibodies so produced can be screen with IgG3 specific antibodies (e.g., BD Pharmigen, CAC-YMS-7635) ensure IgG3 β1AR antibodies are generated.

Various procedures known in the art may be used for the production of polyclonal antibodies directed against β1AR. For the production of antibody, various host animals can be immunized by injection with the peptide corresponding to an β1AR epitope including but not limited to rabbits, mice, rats, sheep, goats, etc. In certain embodiments, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette Guerin) and Corynebacterium parvum)

Monoclonal antibodies against target β1AR are produced by a variety of techniques including conventional monoclonal antibody methodologies such as the somatic cell hybridization techniques of Kohler and Milstein, Nature, 256:495 (1975). Although in some embodiments, somatic cell hybridization procedures are preferred, other techniques for producing monoclonal antibodies are contemplated as well (e.g., viral or oncogenic transformation of B lymphocytes). In general, the preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against β1AR can be generated using transgenic mice carrying the complete human immune system rather than-the mouse system. Splenocytes from the transgenic mice are immunized with the antigen of interest, which are used to produce hybridomas that secrete human mAbs with specific affinities for epitopes from a human protein. (See e.g., Wood et al., WO 91/00906, Kucherlapati et al., WO 91/10741; Lonberg et al., WO 92/03918; Kay et al., WO 92/03917 [each of which is herein incorporated by reference in its entirety]; N. Lonberg et al., Nature, 368:856-859 [1994]; L. L. Green et al., Nature Genet., 7:13-21 [1994]; S. L. Morrison et al., Proc. Nat. Acad. Sci. USA, 81:6851-6855 [1994]; Bruggeman et al., Immunol., 7:33-40 [1993]; Tuaillon et al., Proc. Nat. Acad. Sci. USA, 90:3720-3724 [1993]; and Bruggeman et al. Eur. J. Immunol., 21:1323-1326 [1991]).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies. (See e.g., Sastry et al., Proc. Nat. Acad. Sci. USA, 86:5728 [1989]; Huse et al., Science, 246:1275 [1989]; and Orlandi et al., Proc. Nat. Acad. Sci. USA, 86:3833 [1989]). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and the PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies. (See e.g., Larrick et al., Biotechniques, 11:152-156 [1991]). A similar strategy can also been used to amplify human heavy and light chain variable regions from human antibodies (See e.g., Larrick et al., Methods: Companion to Methods in Enzymology, 2:106-110 [1991]).

Chimeric mouse-human monoclonal antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (See e.g., Robinson et al., PCT/US86/02269; European Patent Application 184,187; European Patent Application 171,496; European Patent Application 173,494; WO 86/01533; U.S. Pat. No. 4,816,567; European Patent Application 125,023 [each of which is herein incorporated by reference in its entirety]; Better et al., Science, 240:1041-1043 [1988]; Liu et al., Proc. Nat. Acad. Sci. USA, 84:3439-3443 [1987]; Liu et al., J. Immunol., 139:3521-3526 [1987]; Sun et al., Proc. Nat. Acad. Sci. USA, 84:214-218 [1987]; Nishimura et al., Canc. Res., 47:999-1005 [1987]; Wood et al., Nature, 314:446-449 [1985]; and Shaw et al., J. Natl. Cancer Inst., 80:1553-1559 [1988]).

The chimeric antibody can be further humanized by replacing sequences of the Fv variable region that are not directly involved in antigen binding with equivalent sequences from human Fv variable regions. General reviews of humanized chimeric antibodies are provided by S. L. Morrison, Science, 229:1202-1207 (1985) and by Oi et al., Bio. Techniques, 4:214 (1986). Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPIIbIIIa antibody producing hybridoma. The recombinant DNA encoding the chimeric antibody, or fragment thereof, can then be cloned into an appropriate expression vector.

Suitable humanized antibodies can alternatively be produced by CDR substitution (e.g., U.S. Pat. No. 5,225,539 (incorporated herein by reference in its entirety); Jones et al., Nature, 321:552-525 [1986]; Verhoeyan et al., Science, 239:1534 [1988]; and Beidler et al., J. Immunol., 141:4053 [1988]). All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method that is capable of replacing at least a portion of a CDR of a human antibody with a CDR derived from a non-human antibody. The human CDRs may be replaced with non-human CDRs; using oligonucleotide site-directed mutagenesis.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

In some embodiments, the monoclonal antibody is a murine antibody or a fragment thereof. In other embodiments, the monoclonal antibody is a bovine antibody or a fragment thereof. For example, the murine antibody can be produced by a hybridoma that includes a B cell obtained from a transgenic mouse having a genome comprising a heavy chain transgene and a light chain transgene fused to an immortalized cell. The antibodies can be full-length IgG3 antibody or can include only an antigen-binding portion (e.g., a Fab, F(ab′)2, Fv or a single chain Fv fragment).

In preferred embodiments, the immunoglobulin is a recombinant antibody (e.g., a chimeric or a humanized antibody), a subunit, or an antigen binding fragment thereof (e.g., has a variable region, or at least a complementarity determining region (CDR)). In some embodiments, the immunoglobulin is monovalent (e.g., includes one pair of heavy and light chains, or antigen binding portions thereof). In other embodiments, the immunoglobulin is a divalent (e.g., includes two pairs of heavy and light chains, or antigen binding portions thereof).

In some embodiments, the present invention provides vaccine compositions comprising an antigenic protein that will generated IgG3 β1AR antibodies in a subject. In certain embodiments, the antigenic protein is at least part of SEQ ID NO:1 (e.g., SEQ ID NO:2). The present invention is not limited by the particular formulation of a vaccine composition. Indeed, a vaccine composition of the present invention may comprise one or more different agents in addition to the fusion protein. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a vaccine composition comprising an antigenic protein comprises an agent and/or co-factor that enhance the ability of the immunogen to induce an immune response (e.g., an adjuvant). In some preferred embodiments, the presence of one or more co-factors or agents reduces the amount of immunogen required for induction of an immune response (e.g., a protective immune response (e.g., protective immunization)). In some embodiments, the presence of one or more co-factors or agents can be used to skew the immune response towards a cellular (e.g., T cell mediated) or humoral (e.g., antibody mediated) immune response. The present invention is not limited by the type of co-factor or agent used in a therapeutic agent of the present invention.

Adjuvants are described in general in Vaccine Design—the Subunit and Adjuvant Approach, edited by Powell and Newman, Plenum Press, New York, 1995. The present invention is not limited by the type of adjuvant utilized (e.g., for use in a composition (e.g., pharmaceutical composition). For example, in some embodiments, suitable adjuvants include an aluminium salt such as aluminium hydroxide gel (alum) or aluminium phosphate. In some embodiments, an adjuvant may be a salt of calcium, iron or zinc, or may be an insoluble suspension of acylated tyrosine, or acylated sugars, cationically or anionically derivatised polysaccharides, or polyphosphazenes.

### EXAMPLES

The following examples are for purposes of illustration only and are not intended to limit the scope of the claims.

### Example 1

### IgG3(+) β1AR Autoantibody for Treating Heart Disease

Dysregulation of autoimmune response triggers the generation of IgG autoantibodies targeting human β1ARs mediating deleterious cardiac outcomes. We investigated the role of β1-adrenergic receptor (β1AR) autoantibodies in human β1AR function. Serum and purified IgG3(+) autoantibodies from patients with onset of cardiomyopathy were tested using HEK 293 cells expressing human β1ARs (HEK-β1AR cells). Unexpectedly, pre-treatment of cells with IgG3(+) serum or purified IgG3(+) β1AR autoantibodies impaired dobutamine-mediated adenylyl cyclase (AC) activity and cAMP generation while enhancing biased β-arrestin recruitment and ERK activation. Contrastingly, β-blocker metoprolol increased AC activity and cAMP in presence of IgG3(+) serum or IgG3(+) β1AR autoantibodies. Since IgG3(+) β1AR autoantibodies are specific to human β1ARs, non-failing human hearts were used as an endogenous system to determine their ability to bias β1AR signaling. Consistently, metoprolol increased AC activity reflecting the ability of the IgG3(+) β1AR autoantibodies to bias β-blocker towards G-protein coupling. Importantly, IgG3(+) β1AR autoantibodies are specific towards β1AR as they do not alter β2AR signaling. Thus, IgG3(+) β1AR autoantibody biases β-blocker towards G-protein coupling, while impairing agonist-mediated G-protein activation but promoting G-protein-independent ERK activation that may underlie the beneficial outcomes observed in patients harboring IgG3(+) β1AR autoantibodies.

### Materials and Methods

### Stable Cell Lines

HEK cells stably overexpressing FLAG-β1AR (FLAG-β1AR-HEK 293) was used in the experiments. Stable cell line was developed in-house by clonal selection using G418 (Geneticin) as antibiotic selection after transfecting HEK 293 cells with mammalian expression vector plasmid [pcDNA3.1(−)] containing Flag tagged—β1AR cDNA (gift from Dr. Yang K. Xiang, UC Davis, Davis, CA). HEK 293 cells stably expressing β2AR (FLAG-β2AR-HEK 293) was a gift from Dr. Robert J. Lefkowitz, Duke University, Durham, NC (Shenoy et al., 2006).

### Human Heart Tissues

De-identified non-failing donor human heart tissues were obtained from Cleveland Clinic's Tissue Bank following approved IRB protocol and consent.

### Cell Culture

For optimal growth of cells, Minimal Essential Media (MEM) supplemented with 10% Fetal Bovine Serum and 5% penicillin-streptomycin was used. Cells were incubated at 37° C. and 5% CO₂.

### Cell Treatments

The cells were serum starved with serum free MEM medium for 4 hours prior to pre-treatment and stimulation. HEK-β1AR and β2AR cells were pre-treated with IgG3(+) or (−) human serum, or affinity purified IgG3(+) or (−) immunoglobulins (autoantibodies) for 30 minutes. Following pre-treatment β1AR cells were treated with specific agonist Dob or specific β1-blocker Metoprolol. HEK-β2AR cells were treated with specific agonist Iso or specific inverse agonist ICI following pre-treatment. The whole cell extracts were prepared by lysing the cells in NP-40 lysis buffer (20 mM Tris-HCl, pH 7.4; 137 mM NaCl; 1% NP40; 20% Glycerol, 1 mM PMSF; 2 μg/mL Leupeptin and 1 μg/mL Aprotinin).

### Immunoblotting

Whole cell lysates were resolved using SDS-PAGE, immunoblotted for respective proteins. The following primary antibody were used for the study; α-Flag (1:2000), α-β-actin (Sigma-Aldrich, 1:25000), α-pERK (Cell Signaling, 1:2000), α-ERK (Cell Signaling, 1:2000), α-pβ2AR (Santacruz Biotechnology, 1:1000). Appropriate HRP conjugated secondary antibody was used and chemiluminescent signals were assessed. Densitometry analysis was performed using image J software.

### Plasma Membrane Isolation

The plasma membrane was isolated using a previously described protocol (Naga Prasad et al., 2001). Briefly the treated cells were scraped using osmotic lysis buffer (5 mM Tris-HCl, pH 7.4; 5 mM EDTA; 1 mM PMSF; 2 μg/mL Leupeptin and 1 μg/mL Aprotinin) and homogenized by douncing. Cardiac tissue samples from non-failing human hearts were homogenized in osmotic lysis buffer using Polytron® homogenizer. This was followed by toggling the samples for 10 mins. The intact cells and nuclei were removed by centrifuging these samples at 2500 rpm for 5 mins. The collected supernatant was centrifuged at 17500 rpm for 30 minutes to obtain the plasma membrane as pellet. All the above steps were performed at 4° C. The pellet was resuspended in ice cold binding buffer (75 mM Tris-HCl, pH 7.5, 2 mM EDTA, and 12.5 mM MgCl₂) to perform receptor binding and adenylate cyclase assay.

### Receptor Binding Assay (βAR Density Assay)

The expression of the βAR receptors on plasma membrane was done using radioligand binding assay using βAR specific radioligand ¹²⁵I-Cyanopindolol (Ferguson et al., 1996; Naga Prasad et al., 2001). This assay was performed by incubating 20 μg of plasma membrane at 37° C. for 1 hour and non-specific binding was assessed in the presence of β1AR specific β-blocker Metoprolol.

cAMP Assay

cAMP generation assay was performed using whole cell lysates. The assay was done using the standard manufacturer's protocol. Catchpoint cAMP fluorescent assay kit from molecular devices (San Jose, CA) was used to measure the cAMP levels (Vasudevan et al., 2013).

### Adenylate Cyclase Assay

G-protein coupling was measured using 20 μg of plasma membrane for adenylate cyclase assay and by measuring the generated cAMP using standard procedure as previously reported (Vasudevan et al., 2011b).

### β-Arrestin2 Recruitment Assay

Measurement of β-arrestin2 recruitment was performed on HTLA cells, which are HEK293 cell line stably expressing a tTA-dependent luciferase reporter and a β-arrestin2-TEV fusion gene (gift from the laboratory of Dr. Bryan L Roth) following protocol as previously described (Kroeze et al., 2015). Briefly, HTLA cells were seeded on a ploy-D-lysine coated 96-well, white wall clear-bottom plate (Corning, #Cat-356651) at a density of ˜30,000 cells/well maintained in 100 μl DMEM supplemented with 10% FBS, 100 U/ml penicillin, 100 μg/ml streptomycin, 2 μg/ml puromycin and 100 μg/ml hygromycin B in a humidified atmosphere at 37° C. in 5% CO2 (day 1). The following day (day 2), cells were transfected with ADRB1-Tango plasmid (Roth Lab PRESTO-Tango GPCR Kit-addgene #Cat 1000000068) using Lipofactamine 3000 transfection kit (Invitrogen, Cat #L3000008). On day 4, human sera negative/positive for IgG3 AAb or affinity purified IgG3(−)/IgG3(+) AAb diluted in assay buffer (20 mM HEPES and 1×HBSS, pH 7.4) was added (10 μl of 10× concentration), to the respective wells of the 96-well plate. Following the incubation for 30 min, 10 μM Dob was added (25 μl of 5× concentration). On day 5, spent medium and drug solutions were removed from the wells by aspiration, and 100 μl of Bright-Glo solution (Promega, #Cat E2620) diluted 5-fold in assay buffer was added to each well. After incubation for 10 min at room temperature, luminescence was measured in a FlexStation 3 Multi-Mode Microplate Reader (Molecular Devices). Relative luminescence units (RLU) were documented and exported into GraphPad Prism for the analysis of data.

### Confocal Microscopy

HEK-β1AR cells transfected with plasmids containing β-arrestin2-GFP fusion gene were plated on poly-L-Lysine coated coverslips, serum starved, pre-treated with No IgG, affinity purified IgG3(−) or IgG3(+) AAb, stimulated with Dob, fixed with 4% paraformaldehyde for 30 min and mounted using prolong gold mountant. Samples were visualized using sequential line excitation at 488 and 405 nm for green and blue respectively. Quantitation of the fluorescence intensity of the cells and plot profile measurements were done using IMAGEJ software.

ELISA and IgG Purification from Plasma Sample

The ELISA and IgG purification has been described in detail previously (Nagatomo et al, 2009, J. Card Fail; Nagatomo et al, 2016, J Card Fail, 22(6):417-422). Briefly, fusion protein containing the second extracellular loop of the human β1AR (amino acids 197 to 222, HWWRAESDEARRCYNDPKCCDFVTNR, SEQ ID NO:2) was synthesized (as the target peptide) and (1 μg) coated on the microtiter plates. 100 μL of patient serum (at dilutions starting from 1:20) was added to the coated wells of the microtiter plate. Then wells were incubated with either IgG-specific (BD Pharmigen, 555788) or IgG3-specific (BD Pharmigen, CAC-YMS-7635) HRP-conjugated antibodies. Following multiple washes the bound horseradish peroxidase-conjugated antibodies were detected via tetramethylbenzidine development (BD Pharmigen, 555214). Color development was stopped with the addition of 2N H₂SO₄. The use of IgG3-specific antibodies allowed differentiation between IgG3+ or IgG3− patient samples. The optical reading was determined at 450 nm and the positive reaction was defined as ≥2.5 times the background. Following the identification of IgG3(+) positive patients samples by ELISA signal, β1AR autoantibodies were purified using a MabTrap Kit (GE #17-1128-01), based on manufacturers protocol. The kit had a binding capacity of 25 mg IgG/mL medium. Briefly the purification began with first collecting the protein from the plasma sample. This was done by adding the collected plasma to a syringe containing 1.5 mL binding buffer (20 mM sodium phosphate, pH 7.0). A 0.45 mM filter was used to flush 1 mL of the binding buffer through the syringe to get a total volume of 2.5 mL. This was followed by purification of the obtained protein, where first the IgG chromatography column was equilibrated with binding buffer. The samples were then slowly added to the column using a syringe at a speed of ˜1 drop/2 sec-10 sec (0.2-1 mL/min). The samples were collected and reloaded back to the column. This step was repeated 4 times. This was followed by washing the samples with binding buffer until no materials appeared in the effluent. The samples were then eluted with approximately 3-5 mL of elution buffer (100 mM glycine-HCl, pH 2.7) and eluate was neutralized in a collection tube containing 375 mL of neutralizing buffer (100 mM Tris-HCl, pH 9.0). The columns were reconditioned using 5 mL binding buffer. The final step was the enrichment of the obtained IgG. The collected samples were passed through a 10 kDa Amicon Ultra tube containing 10 mL of PBS. This was centrifuged at 4000×g for 30 min. Finally, about 200 mL of samples from the above steps were centrifuged at 14000 g for 30 mins using a 3 kDa Amicon Ultra 0.5 ml device to collect the enriched IgG. The enriched IgG3(−) or IgG3(+) autoantibodies were then diluted to a final concentration of 0.5 μg/μl and used for the outlined mechanistic studies.

### Statistical Analysis

All data were expressed as mean±SEM (n≥3 experiments performed under identical conditions). ANOVA was used for multiple comparisons of the data. Statistical analyses were performed using GraphPad Prism© and the significance between the treatments were determined by Student t-test. A p-value less than <0.05 was considered statistically significant.

### Results

### Human Embryonic Kidney 293 (HEK 293) Cells Stably Expressing Human β1AR.

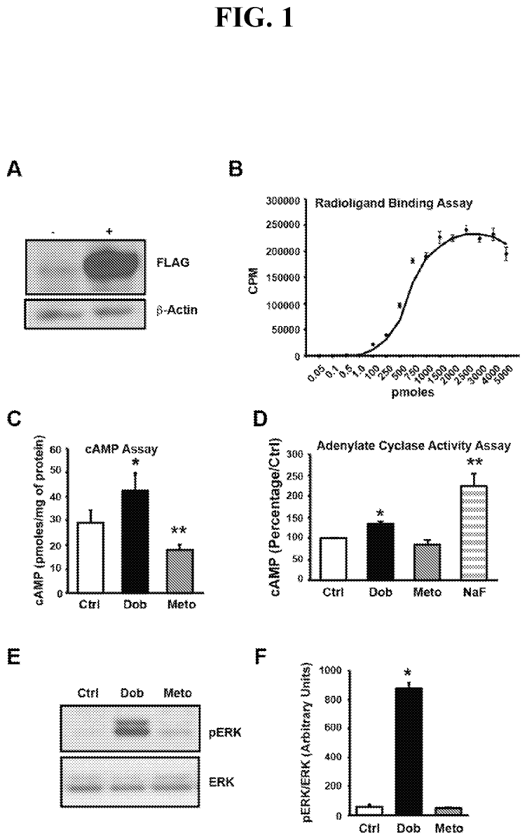

To understand how the IgG3 subclass of β1AR autoantibodies modulate the function of human β1AR, we generated the HEK 293 cell line stably expressing FLAG-tagged human β1AR (β1AR-HEK 293 cells). The expression of the FLAG-β1AR in the cells was tested by western immunoblotting using anti-FLAG antibody. Immunoblotting of cell lysates showed robust expression of FLAG-β1AR in β1AR-HEK 293 cells [FIG. 1A]. Cell surface expression of the β1ARs was confirmed by radio-ligand binding using ^{[125]}I-Cyanopindolol on plasma membranes isolated from β1AR-HEK 293 cells. The binding curve showed high levels of β1AR expression on the cell surface [FIG. 1B]. To determine whether overexpressed β1ARs were functional, cAMP generation was measured in β1AR-HEK 293 cells treated with either the β1AR-specific agonist dobutamine (Dob) or -blocker metoprolol (Meto). The cAMP generation was significantly increased in cells treated with Dob and was dramatically suppressed with Meto, reflecting functional fidelity of overexpressed FLAG-β1ARs [FIG. 1C]. Given that G-protein coupling is a key tenet of β1AR function, we measured G-protein coupling by performing adenylyl cyclase (AC) assay using plasma membrane fractions. Dob treatment significantly increased AC activity which was markedly suppressed with Meto [FIG. 1D]. Sodium fluoride (NaF) was used as positive control to directly stimulate AC and to show the integrity of the G-protein coupling complex in the isolated plasma membranes [FIG. 1D]. To further test for activation of downstream signaling, β1AR-HEK 293 cell lysates were immunoblotted for phospho-ERK (pERK) levels [FIG. 1E]. ERK phosphorylation was significantly increased by Dob stimulation and Meto treatment did not appreciably alter ERK phosphorylation [FIG. 1F]. This human β1AR-overexpressing, HEK 293 cell line now provided the primer to determine the role of the IgG3 subclass of β1AR autoantibodies in modulating β1AR function.

Role of Human Serum Containing β1AR Autoantibodies on cAMP Generation.

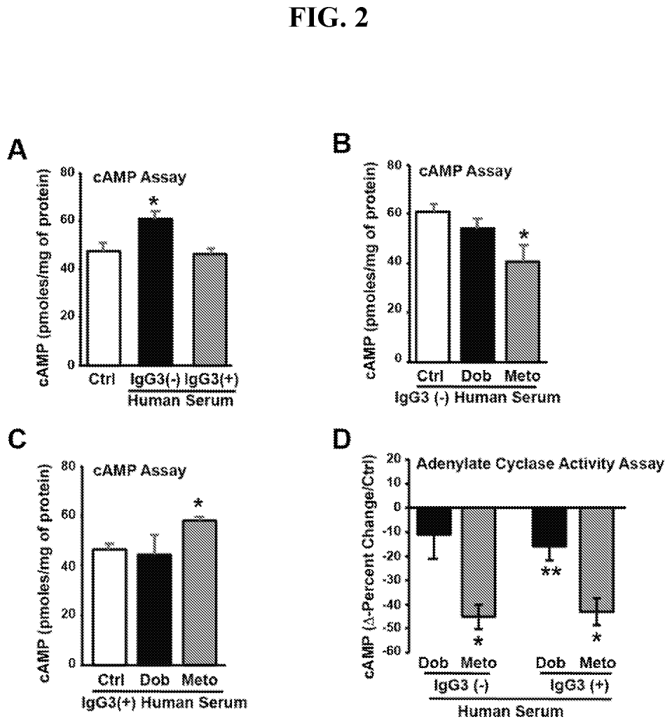

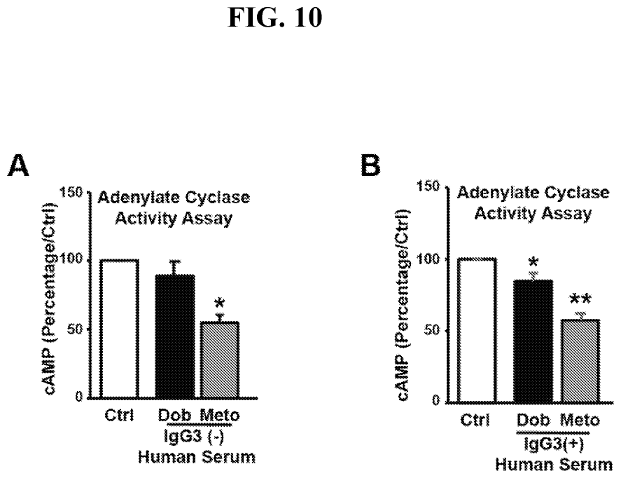

To assess whether β1AR autoantibodies modulate β1AR function, we used the validated β1AR-HEK 293 cells for these studies. Human sera positive for IgG were collected and classified into IgG3(−) and IgG3(+) (classification described elsewhere (Iwata et al., 2001; Nagatomo et al., 2011; Nagatomo et al., 2016; Nagatomo et al., 2017; Nagatomo et al., 2009)). The β1AR-HEK 293 cells were treated with these sera and the cAMP generation measured. Treatment of the cells with IgG3(−) sera significantly increased cAMP whereas, IgG3(+) sera did not alter cAMP levels compared to untreated controls [FIG. 2A]. To test whether IgG3-specific sera containing β1AR autoantibody modulates β1AR responses to Dob or Meto treatment, cells were pre-treated with IgG3(−) or IgG3(+) sera, and cAMP generation was measured following Dob or Meto treatment. Pre-treatment of cells with IgG3(−) sera blunted the Dob-mediated cAMP generation, while Meto significantly decreased cAMP production [FIG. 2B]. Similarly, pre-treatment with IgG(+) sera blocked cAMP generation following Dob [FIG. 2C]. However, surprisingly Meto treatment resulted in significant increase in cAMP generation in IgG3(+) sera treated cells [FIG. 2C]. To further elucidate how serum containing IgG3(+) β1AR autoantibodies allows for differential G-protein coupling, effect of Dob or Meto was assessed by measuring adenylate cyclase (AC) activity (which is a direct measure of G-protein coupling). Pre-treatment of plasma membrane with human serum containing IgG3(−) autoantibodies decreased Dob-mediated AC activity but did not reach significance and significantly reduced AC activity by Meto treatment [FIG. 2D; FIG. 10A]. In contrast, pre-treatment of plasma membranes with human serum containing IgG3(+) immunoglobulins significantly decreased Dob-mediated increase in adenylyl cyclase activity [FIG. 2D; FIG. 10B]. Pre-treatment with human serum containing IgG3(+) autoantibodies significantly decreased AC activity with Meto treatment [FIG. 2D; FIG. 10B].

Role of Affinity Purified β1AR Autoantibodies on cAMP Generation.

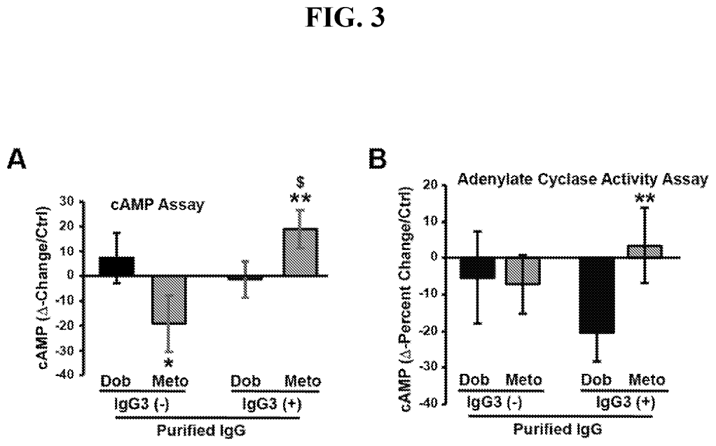

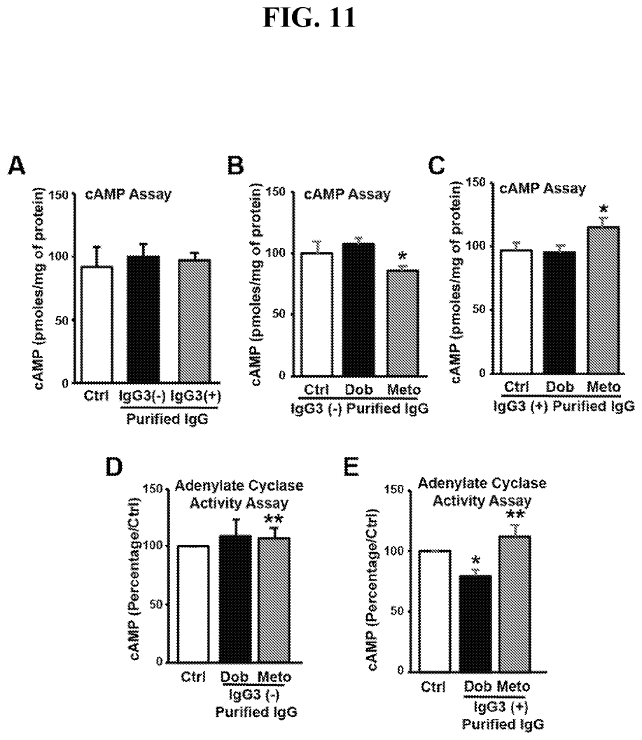

Since sera could contain factors that may potentially regulate β1ARs beyond β1AR autoantibodies, the autoantibodies were affinity-purified to specifically determine their role in modulating Dob- or Meto-mediated signaling. Treatment of cells with the purified immunoglobulins, IgG3(−) or IgG3(+) alone did not alter cAMP generation [FIG. S2A] in contrast to the sera alone treatment [FIG. 2A]. Pre-treatment of cells with affinity purified IgG3(−) immunoglobulins resulted in a slight increase in Dob-mediated cAMP generation and significant decrease in cAMP with Meto [FIG. 3A; FIG. 11B], consistent with IgG3(−) sera [FIG. 2B]. Interestingly, Dob-mediated cAMP generation was abrogated in cells following pre-treatment with affinity purified IgG3(+) immunoglobulins [FIG. 3A; FIG. 11B] suggesting inhibition of G-protein coupling upon agonist binding. Surprisingly, Meto treatment reversed this inhibition resulting in significant generation of cAMP [FIG. 3A; FIG. 11C]. These observations show that both IgG3(−) and IgG3(+) samples have divergent effects with Dob-mediated or Meto-mediated cAMP generation. Importantly, presence of IgG3(+) immunoglobulins uniquely modulates β1AR blocker metoprolol to engage in G-protein pathways while contrastingly blocking the classical coupling of G-protein by β1AR agonist dobutamine. To further understand how purified IgG3(+) β1AR autoantibodies allow for differential G-protein coupling, AC activity was assessed following Dob or Met. Dob treatment in the presence of purified IgG3(−) autoantibodies increased AC activity but did not reach significance [FIG. 3B; FIG. 11D]. However, purified IgG3(−) immunoglobulins significantly increased AC activity with Meto treatment [FIG. 3B; FIG. 11D]. This is in contrast to cAMP data wherein pre-treatment with IgG3(−) β1AR autoantibodies resulted in a modest Dob-mediated change in cAMP that did not reach significance, while cAMP levels were significantly reduced in the presence of Meto [FIG. 2D]. However, Meto treatment in the presence of purified IgG3(+) β1AR autoantibodies showed restoration of adenylyl cyclase activity [FIG. 3B; FIG. 11E] compared to the plasma membranes that were pretreated with human serum containing IgG3(+) autoantibodies [FIG. 2D]. The adenylyl cyclase data is in consistent with increase in cAMP generation following treatment with the IgG3(+) human serum [FIG. 2C] or affinity purified IgG3(+) immunoglobulins [FIG. 3A]. These observations strengthen the findings that IgG3(+) β1AR autoantibodies impair Dob-mediated G-protein coupling, while surprisingly allowing Meto, a β-blocker to mediate signals through engagement of G-protein coupled pathways.

### IgG3(+) Autoantibodies Promote β-Arrestin-Bias to β1AR-Agonist

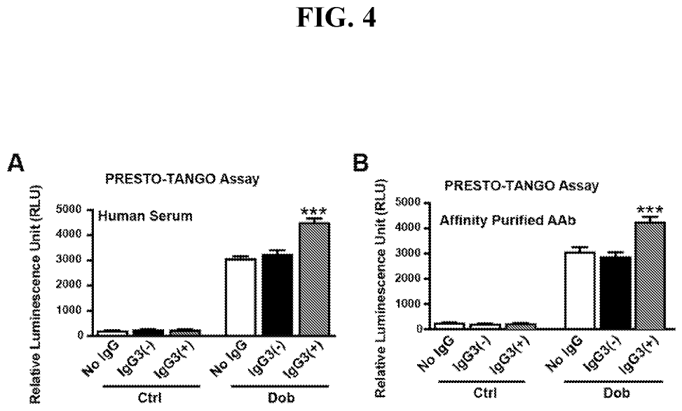

To test whether reduced G-protein coupling to β1AR agonist in the presence of IgG3(+) β1AR antibodies is due to enhanced engagement of scaffolding protein β-arrestin, PRESTO-Tango β-arrestin recruitment assays were performed in HTLA cells (HEK cells expressing tTA-dependent luciferase reporter and a β-arrestin2-TEV fusion gene). The treatment of cells with human sera harboring either IgG3(−) or IgG3(+) autoantibodies did not alter the β-arrestin engagement (as measured by relative luminescence) to β1AR complex [FIG. 4A]. However, Dob treatment of these cells resulted in significant increase of luminescence specifically in cells pre-treated with human sera containing IgG3(+) autoantibodies reflecting the unique β-arrestin bias promoted by IgG3(+) autoantibodies. To directly test the role IgG3(+) auto-antibodies in promoting β-arrestin bias, the PRESTO-Tango assay was performed using affinity purified autoantibodies. Treatment of cells with IgG3(−) or IgG3(+) β1AR autoantibodies by itself did not result in any significant recruitment of β-arrestin to β1AR complex as reflected by no appreciable changes in luminescence [FIG. 4B]. Notably, pretreatment of cells with IgG3(+) β1AR autoantibodies significantly increased Dob-mediated recruitment of β-arrestin to the receptor complex compared to no IgG or IgG3(−) autoantibodies [FIG. 4B].

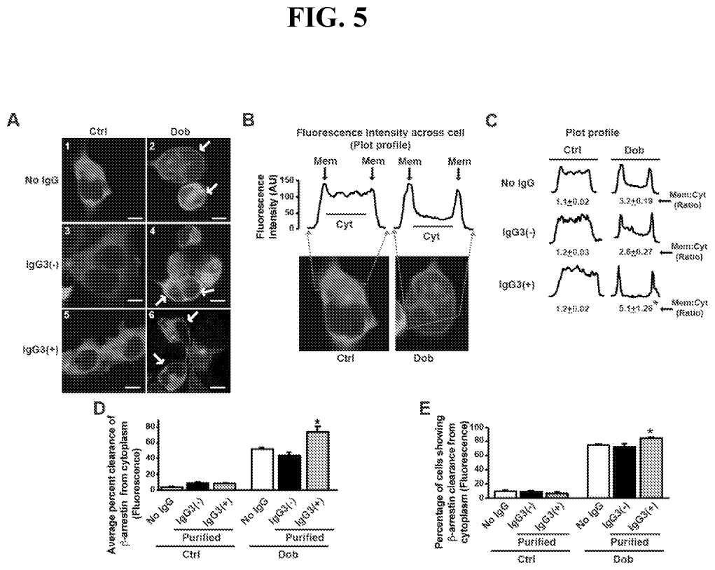

To further validate the role of IgG3(+) autoantibodies in biasing β1AR agonist towards arrestin, β-arrestin recruitment assays were performed. β1AR-HEK cells were transiently transfected with β-arrestin2-GFP fusion construct to visualize the β-arrestin recruitment to the β1ARs using confocal microscropy. The cells were pre-treated with IgG3(−) or IgG3(+) autoantibodies and stimulated with Dob wherein, no IgG treatment served as a control. The clearance of green fluorescence from the cytoplasm is used as a measure of β-arrestin recruitment [FIG. 5]. Pre-treatment of cells with either IgG3(−) [FIG. 5A; panel 2] or IgG3(+) autoantibodies [FIG. 5A; panel 3] did not alter the β-arrestin recruitment and was similar to No IgG control [FIG. 5A; panel 1]. Dob-mediated β-arrestin recruitment was significantly increased when cells were pretreated with IgG3(+) autoantibodies [FIG. 5A; panel 6] compared to the cells pretreated with IgG3(−) autoantibodies [FIG. 5A, panel 5] or No IgG [FIG. 5A; panel 4]. Given the quantitative nature of increased β-arrestin recruitment to the β1AR complex following Dob in presence of IgG(+) versus IgG(−) autoantibody, we performed comprehensive plot profiles of the GFP fluorescence across the cells to assess the ratio of membrane (Mem) to cytoplasm (Cyt) fluorescence intensity across various cells [FIG. 5B]. Consistent with Dob mediating β-arrestin recruitment to β1AR complex, cells pre-treated with No IgG or IgG3(−) auto-antibodies showed β-arrestin clearance from the cytoplasm in significant percentage of GFP expressing cells compared to no Dob treatment [FIG. 5C upper & middle panel]. Plot analysis summary showed that pre-treatment of cells with IgG3(+) auto-antibodies resulted in significantly higher changes in the fluorescence intensity (represented as percentage) characterized by cytoplasmic clearance of β-arrestin compared to No IgG or IgG3(−) autoantibodies [FIG. 5D]. Furthermore, pre-treatment with IgG3(+) autoantibodies resulted in significant percentage of cells showing changes in the total cell fluorescence (a reflection of loss in cytoplasmic fluorescence due to β-arrestin recruitment to the membrane) compared to No IgG or IgG3(−) autoantibodies [FIG. 5E]. These findings show that the human IgG3(+) β1AR autoantibodies blunt G-protein coupling and selectively enhance β-arrestin bias in response to β1AR agonist which may underlie their beneficial outcomes.

### IgG3(+) β1AR Autoantibodies Modulate Downstream ERK Activation.

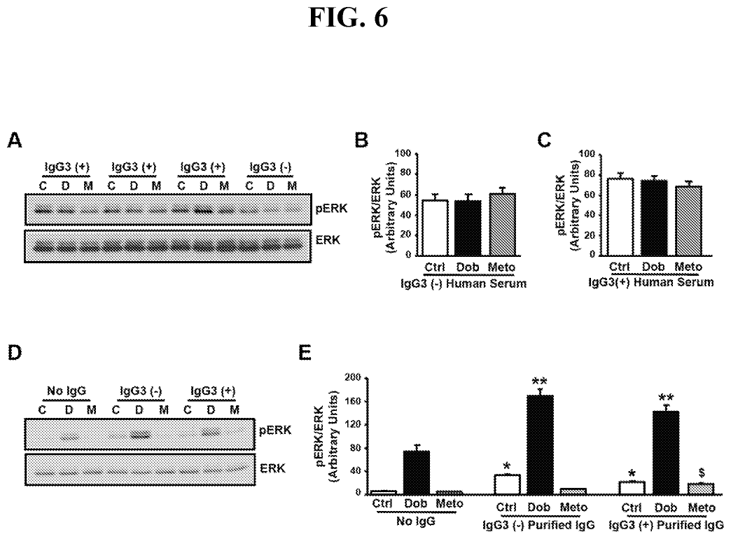

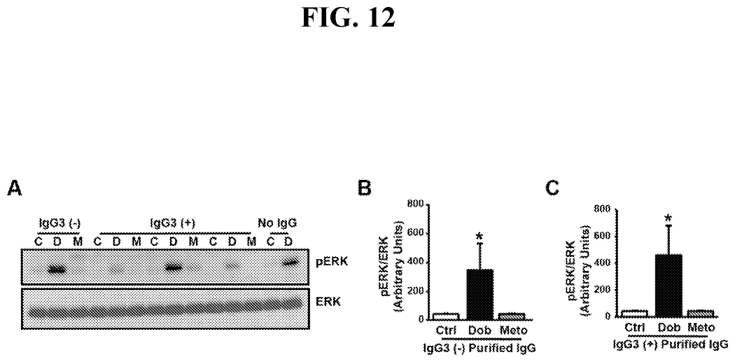

Activation of ERK is one of the key measures of biased downstream G-protein independent signaling that is mediated by β-arrestins (Shenoy et al., 2006). Since IgG3(+) β1AR autoantibodies block G-protein coupling upon Dob, we assessed for changes in phospho-ERK following IgG3(−) or IgG3(+) sera or affinity purified antibodies. Treatment of cells with either IgG3(+) or IgG3(−) serum by itself did not result in any significant difference in ERK activation [FIG. 6 A, B & C]. However, Dob or Meto treatment of cells in the presence of IgG3(−) or IgG3(+) sera showed no appreciable ERK activation [FIG. 6 A, B & C]. These data suggest that both IgG3(−) or IgG3(+) sera impairs Dob mediated ERK activation while Meto does not alter phospho-ERK status. Since sera may contain components that could alter β1AR function and signaling, we tested for ERK activation using affinity purified β1AR autoantibodies. Treatment of both IgG3(−) and IgG3(+) autoantibodies activated ERK and surprisingly Dob-mediated ERK phosphorylation was restored in cells pre-treated with affinity purified IgG3(−) or IgG3(+) β1AR autoantibodies [FIG. 6 D & E; FIG. 12 A, B & C]. However, Meto treatment did not activate ERK in the presence of IgG3(−) affinity purified β1AR autoantibodies [FIG. 6 D, E & F; FIG. 12 A, B & C] but Meto treatment significantly activated ERK in the presence of IgG3(+) autoantibodies. It is important to note that IgG3(+) autoantibodies that abrogates G-protein coupling with Dob [FIG. 3B] does not impair ERK activation while Meto does activate ERK. This suggests that IgG3(+) autoantibodies could uniquely bias the agonist signaling towards pathways reflecting a yet unappreciated role of IgG3(+) autoantibodies in regulating receptor function.

### β1AR Autoantibodies Alter AC Activity in Human Cardiac Membranes.

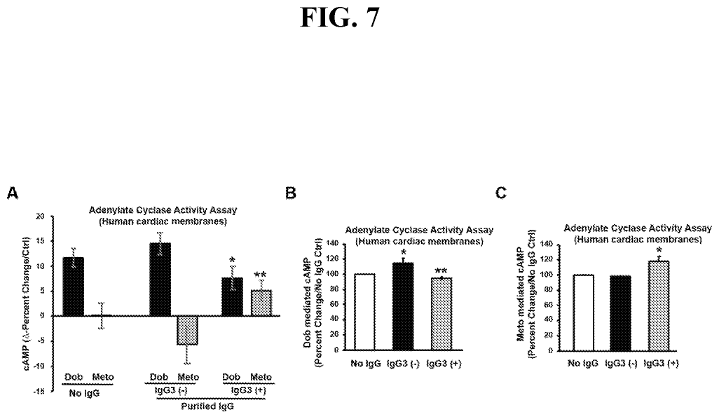

In order to understand if the observations made using heterologous cells system is physiologically relevant, non-failing donor human hearts were used to isolate cardiac plasma membranes to determine AC activity in presence or absence of purified IgG3 autoantibodies. Dob-mediated significant decrease of AC activity in the plasma membranes pretreated with IgG3(+) autoantibodies when compared to both IgG3(−) autoantibodies and no IgG control [FIGS. 7A & B]. However, divergent AC activity was observed in response to Meto following pre-treatment with IgG3(−) or IgG3(+) autoantibodies. Meto mediated inhibition of AC activity in presence of with IgG3(−) autoantibodies [FIGS. 7A & C]. In contrast, presence of IgG3(+) autoantibodies resulted in significant increase in AC activity [FIGS. 7A & C] reflecting their ability to mediate opposing effects selectively in response to β1-blocker metoprolol. In the absence of IgG3 autoantibodies, the cardiac plasma membranes from non-failing human hearts responded to classically to agonist Dob by activating AC activity and to Met by inhibiting AC activity [FIG. 7A]. The inhibition of AC activity in the presence of 3-agonist and increase of AC activity in the presence β-blocker of when pre-treated with IgG3(+) autoantibodies shows a unique ability of this sub-class of autoantibodies to preserve P1 AR function that may underlie the benefits observed in patients harboring these autoantibodies (Nagatomo et al., 2017).

### β1AR Autoantibodies do not Alter β2AR Signaling.

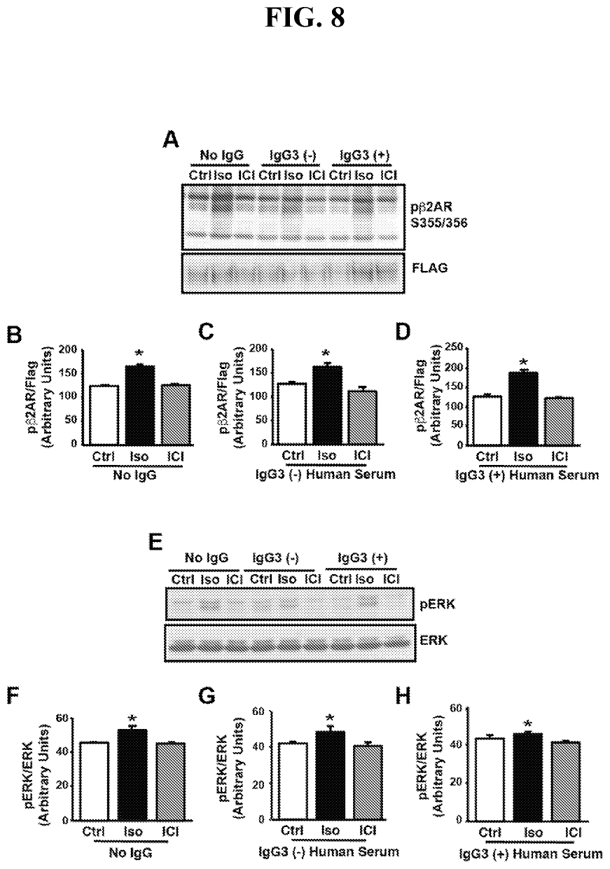

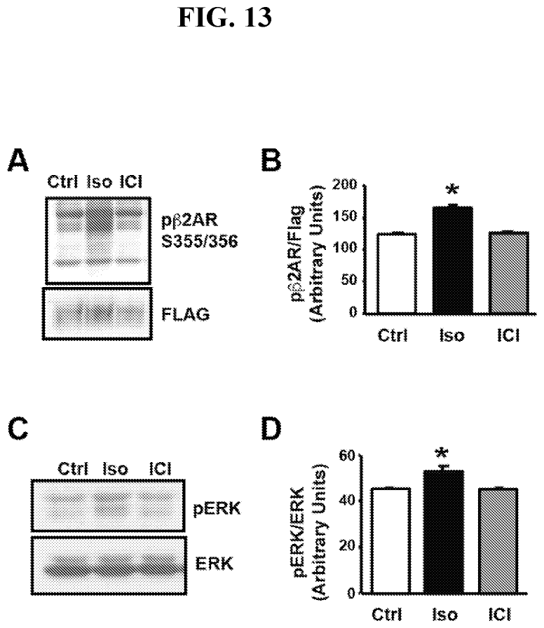

To test whether β1AR autoantibodies are specific to only modulating β1AR signaling, we used HEK 293 cells stably expressing β2AR (β2AR-HEK 293 cells) [FIG. 13) (Shenoy et al., 2006; Vasudevan et al., 2011b; Vasudevan et al., 2013). β2AR-HEK 293 cells were pretreated with either no sera, IgG3(−) or IgG(+) sera. Following pre-treatment the cells were stimulated with isoproterenol (ISO, βAR agonist) or ICI (an inverse β2AR antagonist) and immunoblotting was performed to assess phospho-32AR as a measure of activation. There was significant increase in β2AR phosphorylation following ISO treatment with either no sera, IgG3(−) or IgG(+) sera [FIG. 8 A, B, C & D]. Similarly, IgG(−) or IgG(+) sera did not alter phosphorylation state of 32ARs upon ICI treatment [FIG. 8A upper panel, B, C & D]. These studies show that IgG3(−) or IgG(+) sera do not alter β2AR responses to ISO. FLAG immunoblotting was performed as loading control [FIG. 8A lower panel]. To further show that IgG(−) or IgG(+) sera does not alter downstream signaling, phospho-ERK was assessed following pre-treatment with sera and stimulation with ISO or ICI. Pre-treatment with either IgG3(−) or IgG3(+) sera did not affect ISO-mediated ERK activation or alter ICI dependent responses [FIG. 8 E, F, G & H]. Together these data suggest that sera containing β1AR autoantibodies from either the IgG3(−) or IgG3(+) family of immunoglobulins does not affect the activation/downstream signaling of β2ARs reflecting the specificity of these autoantibody to selectively modulate human β1AR signaling.

β1AR autoantibodies have been found in 30-95% of patients with a diagnosis of idiopathic dilated cardiomyopathy (DCM) (Limas et al., 1992; Wallukat et al., 1991) and higher percentages are consistently associated with patients requiring left ventricular assist device (LVAD) (Dandel et al., 2012; Youker et al., 2014). Such a co-relation suggests a role for β1AR autoantibodies in the progression of heart failure. However, recent studies on patients enrolled in the IMAC-2 (Intervention in Myocarditis and Acute Cardiomyopathy-2) study found favorable myocardial outcomes in patients with β1AR autoantibodies belonging to the IgG3 subclass (Nagatomo et al., 2016; Nagatomo et al., 2017). This suggests the IgG3 subclass of β1AR autoantibodies may uniquely regulate β1AR function, providing benefits compared to the known deleterious role of the IgG class of β1AR autoantibodies. Using IgG3(−) or IgG3(+) β1AR autoantibody-containing sera and affinity-purified β1AR autoantibodies, we show that the IgG3(+) subclass of the antibodies blunt β1AR response to the β1AR agonist, dobutamine by enhancing the recruitment of β-arrestin. However, the IgG3(+) subclass of β1AR autoantibodies modulates the β1AR-blocker, Meto dependent signaling. Surprisingly IgG3(+) β1AR autoantibodies increases adenylyl cyclase activity and cAMP levels with metoprolol treatment, suggesting a yet, unappreciated role of the IgG3(+) subclass of β1AR autoantibodies that could now bias the Meto signal endogenously. Such an unique role for the IgG3(+) human β1AR autoantibodies in modulating β1AR G-protein coupling by Meto was validated using cardiac plasma membranes from non-failing donor human heart sample that endogenously harbors β1ARs. This unique signaling mechanism may potentially underlie the beneficial outcomes observed in patients.

Autoantibodies classically belong to the IgG class of immunoglobulins which are one of the abundant among the five classes (IgM, IgE, IgG, IgA and IgD) found in human serum. Previous studies have identified that β1AR autoantibodies belongs to IgG class of immunoglobulins can modulate human β1AR responses (Iwata et al., 2001; Jahns et al., 1999a; Jahns et al., 1999b; Kaya et al., 2012). However, the use of the IgG class of β1AR autoantibodies isolated from patients with DCM showed varied responses in terms of their ability to modulate β1AR internalization and cAMP generation (Bornholz et al., 2013). The IgG class of immunoglobulins are further divided into four sub-classes namely, IgG1, IgG2, IgG3 and IgG4 (Schur, 1988; Vidarsson et al., 2014). Thus, the variability in the responses observed to IgG isolates from different patients could potentially be due to changes in relative abundance of these subclasses which may differentially alter β1AR responses and cardiac pathogenic outcomes. Consistent with this concept, recent studies show that patients who harbor the subclass of IgG3 enriched β1AR autoantibodies have significant myocardial recovery compared to the non-IgG3(+) patients (Nagatomo et al., 2017). This suggests that heart failure progression and exacerbation in the patients may depend on the representation of the IgG subclass of β1AR autoantibody.

Previous cellular studies show that pre-treatment with many of the β1AR autoantibody IgG isolates from patients positively modulates agonist isoproterenol (ISO) coupling as measured by cAMP (Bornholz et al., 2013). This suggests that the β1AR autoantibody is able to mediate a receptor conformation that allows for elevated generation of cAMP beyond the levels generated by ISO alone (Bornholz et al., 2013). This supported the concept that IgG class of β1AR autoantibodies may lead to chronic elevation in cAMP levels due to hyper-sympathetic state exacerbating the heart failure outcomes (Iwata et al., 2001; Jahns et al., 1999b; Kaya et al., 2012). In contrast, our studies show that sera containing IgG(+) β1AR autoantibodies or affinity purified IgG3(+) β1AR autoantibodies impair agonist-mediated activation β1ARs as measured by cAMP generation and adenylyl cyclase activity. This suggests that presence of the IgG3 subclass of β1AR autoantibodies may chronically reduce cAMP levels that underlie beneficial outcomes of myocardial recovery observed in these set of patients (Nagatomo et al., 2017). This supports the evolving idea that subclass members (IgG1, IgG2, IgG3 and IgG4) of the IgG family of β1AR autoantibodies may have differential effects on β1AR function as described by our studies on the IgG3 subclass of β1AR autoantibodies and substantiated by studies showing variable cAMP response to pan IgG β1AR autoantibodies (Bornholz et al., 2013).

β-blocker's are known to block βAR responses to agonist and have been identified by their ability to inhibit generation of cAMP following agonist treatment (Kenakin, 2004; Wisler et al., 2007). Since autoantibodies mediate a β1AR confirmation that drives the receptors to generate cAMP in response to agonist (Bornholz et al., 2013), we used metoprolol, a β1AR selective blocker (antagonist) (Bristow, 1997; Prakash and Markham, 2000). It has been recognized that β-blockers alprenolol or carvedilol can bias the downstream β1AR signaling towards G-protein independent ERK activation, while simultaneously block G-protein coupling (Kim et al., 2008; Shenoy et al., 2006; Wisler et al., 2007). While Meto by itself blocks G-protein coupling, the presence of IgG3(+) β1AR autoantibodies surprisingly results in G-protein coupling and cAMP generation. More intriguingly, IgG3(+) β1AR autoantibodies markedly impair agonist dobutamine-mediated G-protein coupling by selectively enhancing the binding of β-arrestin to the β1AR, desensitizing the receptor, while remarkably preserving ERK activation. This observation suggests that IgG3(+) β1AR autoantibodies uniquely biases that agonist signaling towards G-protein independent pathway while blocking the G-protein dependent pathway. In this context, recent studies have shown that b-blocker carvedilol mediates a unique β1AR conformation that selectively allows for Gi coupling promoting G-protein independent 3-arrestin dependent pathway (Wang et al., 2017). Thus, it is possible that IgG3(+) β1AR autoantibodies may bind to the β1ARs promoting Gi coupling and β-arrestin signaling that may underlie the beneficial outcomes observed in patients harboring IgG3(+) β1AR autoantibodies (Nagatomo et al., 2017).

Patients harboring the IgG class of β1AR autoantibodies are known to have worse patient outcomes (Iwata et al., 2001; Jahns et al., 1999b; Kaya et al., 2012), but the contrasting observation of benefits observed with IgG3 subclass suggests differential modulation/engagement of β1ARs to mediate beneficial signals (Nagatomo et al., 2017). The unique ability of the IgG3 subclass of β1AR autoantibodies to bias β1AR signaling through the G-protein-independent ERK pathway may underlie the beneficial outcomes in patients with this antibody (Nagatomo et al., 2017). It is known that β-blockers inhibit G-protein coupling, but also simultaneously initiates signals through β-arrestin-dependent, G-protein independent mechanisms (Kim et al., 2008; Shenoy et al., 2006; Wisler et al., 2007). Since 3-blockers treatment has positive outcomes in heart failure patients, the biased G-protein-independent, β-arrestin-dependent signaling initiated by β-blockers is considered beneficial (Kim et al., 2008; Wisler et al., 2007). However, uniqueness of the IgG3(+) β1AR autoantibodies lies in their ability to modulate β1AR signaling to inhibit the classical agonist-mediated G-protein coupling, while mediating beneficial β-arrestin pathway preserving G-protein-independent ERK activation. Given the hyper-sympathetic state of patients with heart failure, the presence of the IgG3(+) β1AR autoantibodies would allow for preferential engagement of the G-protein-independent pathway, reflecting in the myocardial recovery of patients harboring this sub-class of β1AR autoantibodies (Nagatomo et al., 2017).

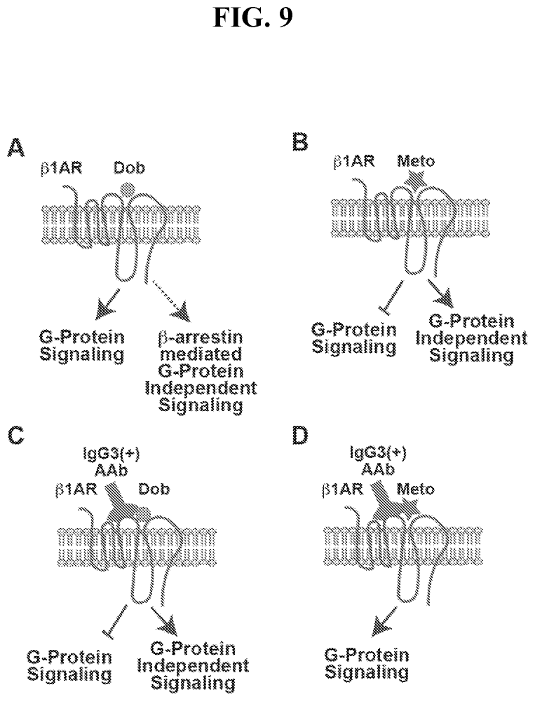

Although one of the limitations of the study is the availability of IgG3(+) patient samples, we still have provided direct G-protein coupling evidence to demonstrate the distinctive ability of the IgG3(+) sub-class of the β1AR autoantibody in modulating β1AR signaling [FIG. 9]. Another limitation in our study is the availability of human tissues/cells endogenously expressing human β1ARs as the auto-antibodies have selectivity towards human β1ARs. Comprehensive protein expression pattern analysis using GeneCard showed appreciable expression of β1ARs in human kidney, brain and heart. Therefore, we have used non-failing human heart samples to show that the human IgG3(+) β1AR autoantibodies uniquely modulate human cardiac β1AR signaling to β1-blocker Meto, an organ whose functional outcome are directly affected by the presence of these sub-class of auto-antibodies.

These observations tempts us to speculate about the unique beneficial modulatory role IgG3(+) β1AR autoantibodies may have in patients with cardiomyopathy. Thus, IgG3(+) β1AR autoantibodies in the presence of high levels of circulating β-blocker would allow for moderate coupling to G-proteins while, with higher levels of circulating catecholamines they would impair G-protein coupling but importantly allow for G-protein independent signaling. These signaling outcomes modulated by IgG3(+) β1AR autoantibodies could have physiological significance given that G-protein independent signaling is considered beneficial in conditions of sympathetic overdrive (Carr et al., 2016; Noma et al., 2007).

### Example 2

### Association of Beta-1 Adrenergic Receptor Autoantibodies with Heart Failure Risk in Systemic Sclerosis

Systemic sclerosis (SSc) is associated with the greatest independent risk of heart failure (HF) among systemic autoimmune diseases. Cardiac involvement in SSc also includes cardiomyopathy (CM), diastolic dysfunction (DD), and arrhythmias. Diffuse cutaneous SSc (dcSSc) and SSc sine scleroderma (ssSSc) are associated with greater cardiac risk than limited cutaneous SSc (lcSSc). We aimed to assay for the presence of these autoantibodies in an SSc cohort.

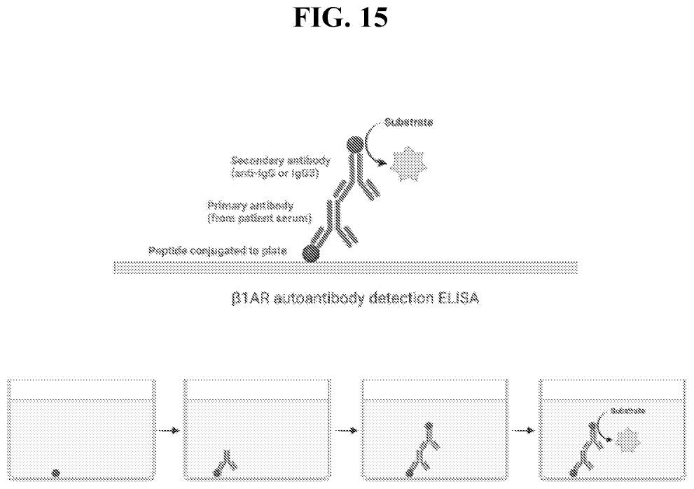

Methods: Serum samples were collected with informed consent from SSc patients with and without cardiac involvement. Enzyme-linked immunosorbent assays (ELISAs) were performed to investigate the presence of circulating IgG and IgG3 subtype β1AR-AAbs (see FIG. 15). Average optical density (OD) was compared between SSc and healthy control samples to establish seropositivity at the 2× control threshold level. Proportions of patients positive for β1AR-AAbs in different groups were compared using Fisher's exact test.

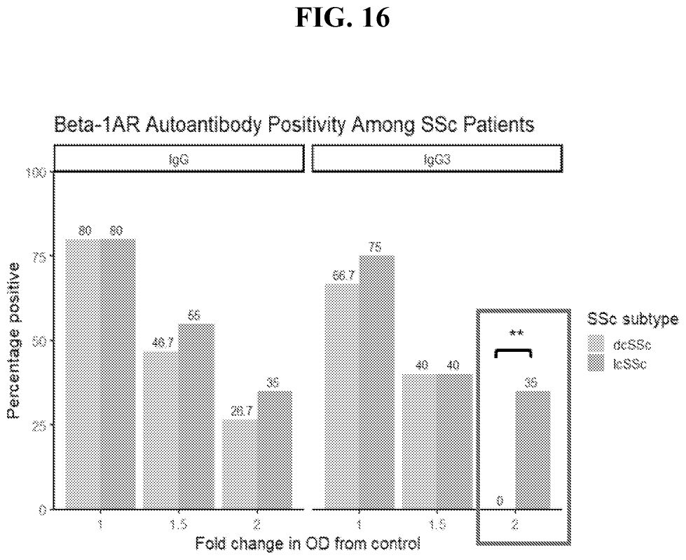

Results: Samples were collected from 36 SSc patients, of whom 20 (56%) had lcSSc, 15 (42%) had dcSSc and 1 (2%) had ssSSc. Cardiac involvement included HF, CM, DD, and arrythmia, at least one of which was documented in 9 patients (25%). Of the total SSc cohort, 11 (31%) and 7 (19%) were positive for anti-β1AR IgG and IgG3 respectively (see FIG. 16). Between SSc patients with and without cardiac involvement, there was no significant difference in IgG or IgG3 positivity (FIG. 16). There was a significant increase in IgG3 positivity in the lcSSc group compared to the dcSSc plus ssSSc group at the 2× control threshold (p=0.011) (FIG. 16).

Conclusion: High anti-β1AR IgG3 positivity is associated with less severe disease category (lcSSc vs dcSSc/ssSSc) in our SSc cohort, supporting prior evidence of the protective role of the IgG3 subtype. Supports prior evidence of the protective role of the IgG3 subtype of anti-β1AR antibodies in HF and evidence of an autoimmune mechanism of myocardial involvement in SSc.

### REFERENCES

Baba, A. (2010). Targeted autoantibodies in apheresis treatment against severe heart failure. Jap J Apheresis 29, 187-193.
Bomholz, B., Weidtkamp-Peters, S., Schmitmeier, S., Seidel, C. A., Herda, L. R., Felix, S. B., Lemoine, H., Hescheler, J., Nguemo, F., Schafer, C., et al. (2013). Impact of human autoantibodies on beta1-adrenergic receptor conformation, activity, and internalization. Cardiovascular research 97, 472-480.
Bristow, M. R. (1997). Mechanism of action of beta-blocking agents in heart failure. Am J Cardiol 80, 26L-40L.
Carr, R., 3rd, Schilling, J., Song, J., Carter, R. L., Du, Y., Yoo, S. M., Traynham, C. J., Koch, W. J., Cheung, J. Y., Tilley, D. G., et al. (2016). beta-arrestin-biased signaling through the beta2-adrenergic receptor promotes cardiomyocyte contraction. Proc Natl Acad Sci USA 113, E4107-4116.
Christ, T., Wettwer, E., Dobrev, D., Adolph, E., Knaut, M., Wallukat, G., and Ravens, U. (2001). Autoantibodies against the beta1 adrenoceptor from patients with dilated cardiomyopathy prolong action potential duration and enhance contractility in isolated cardiomyocytes. Journal of molecular and cellular cardiology 33, 1515-1525.
Crampton, S. P., Voynova, E., and Bolland, S. (2010). Innate pathways to B-cell activation and tolerance. Ann N Y Acad Sci 1183, 58-68.
Dandel, M., Wallukat, G., Englert, A., Lehmkuhl, H. B., Knosalla, C., and Hetzer, R. (2012). Long-term benefits of immunoadsorption in beta(1)-adrenoceptor autoantibody-positive transplant candidates with dilated cardiomyopathy. Eur J Heart Fail 14, 1374-1388.
Deubner, N., Berliner, D., Schlipp, A., Gelbrich, G., Caforio, A. L., Felix, S. B., Fu, M., Katus, H., Angermann, C. E., Lohse, M. J., et al. (2010). Cardiac beta1-adrenoceptor autoantibodies in human heart disease: rationale and design of the Etiology, Titre-Course, and Survival (ETiCS) Study. Eur J Heart Fail 12, 753-762.
Dungen, H. D., Dordevic, A., Felix, S. B., Pieske, B., Voors, A. A., McMurray, J. J. V., and Butler, J. (2020). beta1-Adrenoreceptor Autoantibodies in Heart Failure: Physiology and Therapeutic Implications. Circulation Heart failure 13, e006155.
Engelhardt, S., Hein, L., Wiesmann, F., and Lohse, M. J. (1999). Progressive hypertrophy and heart failure in beta1-adrenergic receptor transgenic mice. Proc Natl Acad Sci USA 96, 7059-7064.
Ferguson, S. S., Downey, W. E., 3rd, Colapietro, A. M., Barak, L. S., Menard, L., and Caron, M. G. (1996). Role of beta-arrestin in mediating agonist-promoted G protein-coupled receptor internalization. Science 271, 363-366.
Iwata, M., Yoshikawa, T., Baba, A., Anzai, T., Mitamura, H., and Ogawa, S. (2001). Autoantibodies against the second extracellular loop of beta1-adrenergic receptors predict ventricular tachycardia and sudden death in patients with idiopathic dilated cardiomyopathy. Journal of the American College of Cardiology 37, 418-424.
Jahns, R., Boivin, V., Siegmund, C., Boege, F., Lohse, M. J., and Inselmann, G. (1999a). Activating beta-1-adrenoceptor antibodies are not associated with cardiomyopathies secondary to valvular or hypertensive heart disease. Journal of the American College of Cardiology 34, 1545-1551.
Jahns, R., Boivin, V., Siegmund, C., Inselmann, G., Lohse, M. J., and Boege, F. (1999b). Autoantibodies activating human beta1-adrenergic receptors are associated with reduced cardiac function in chronic heart failure. Circulation 99, 649-654.
Kaya, Z., Leib, C., and Katus, H. A. (2012). Autoantibodies in heart failure and cardiac dysfunction. Circ Res 110, 145-158.
Kenakin, T. (2004). Principles: receptor theory in pharmacology. Trends Pharmacol Sci 25, 186-192.
Kim, I. M., Tilley, D. G., Chen, J., Salazar, N. C., Whalen, E. J., Violin, J. D., and Rockman, H. A. (2008). Beta-blockers alprenolol and carvedilol stimulate beta-arrestin-mediated EGFR transactivation. Proc Natl Acad Sci USA 105, 14555-14560.
Kroeze, W. K., Sassano, M. F., Huang, X. P., Lansu, K., McCorvy, J. D., Giguere, P. M., Sciaky, N., and Roth, B. L. (2015). PRESTO-Tango as an open-source resource for interrogation of the druggable human GPCRome. Nat Struct Mol Biol 22, 362-369.
Liaudet, L., Calderari, B., and Pacher, P. (2014). Pathophysiological mechanisms of catecholamine and cocaine-mediated cardiotoxicity. Heart Fail Rev 19, 815-824.
Limas, C. J., Goldenberg, I. F., and Limas, C. (1992). Assessment of immune modulation of beta-adrenergic pathways in human dilated cardiomyopathy: influence of methodologic factors. Am Heart J 123, 967-970.
Magnusson, Y., Marullo, S., Hoyer, S., Waagstein, F., Andersson, B., Vahlne, A., Guillet, J. G., Strosberg, A. D., Hjalmarson, A., and Hoebeke, J. (1990). Mapping of a functional autoimmune epitope on the beta 1-adrenergic receptor in patients with idiopathic dilated cardiomyopathy. J Clin Invest 86, 1658-1663.
Magnusson, Y., Wallukat, G., Waagstein, F., Hjalmarson, A., and Hoebeke, J. (1994). Autoimmunity in idiopathic dilated cardiomyopathy. Characterization of antibodies against the beta 1-adrenoceptor with positive chronotropic effect. Circulation 89, 2760-2767.
Mann, D. L. (2011). The emerging role of innate immunity in the heart and vascular system: for whom the cell tolls. Circ Res 108, 1133-1145.
Milano, C. A., Allen, L. F., Rockman, H. A., Dolber, P. C., McMinn, T. R., Chien, K. R., Johnson, T. D., Bond, R. A., and Lefkowitz, R. J. (1994). Enhanced myocardial function in transgenic mice overexpressing the beta 2-adrenergic receptor. Science 264, 582-586.
Naga Prasad, S. V., Barak, L. S., Rapacciuolo, A., Caron, M. G., and Rockman, H. A. (2001). Agonist-dependent recruitment of phosphoinositide 3-kinase to the membrane by beta-adrenergic receptor kinase 1. A role in receptor sequestration. J Biol Chem 276, 18953-18959.
Nagatomo, Y., Baba, A., Ito, H., Naito, K., Yoshizawa, A., Kurita, Y., Nakamura, I., Monkawa, T., Matsubara, T., Wakabayashi, Y., et al. (2011). Specific immunoadsorption therapy using a tryptophan column in patients with refractory heart failure due to dilated cardiomyopathy. J Clin Apher 26, 1-8.
Nagatomo, Y., Li, D., Kirsop, J., Borowski, A., Thakur, A., and Tang, W. H. (2016). Autoantibodies Specifically Against beta1 Adrenergic Receptors and Adverse Clinical Outcome in Patients With Chronic Systolic Heart Failure in the beta-Blocker Era: The Importance of Immunoglobulin G3 Subclass. J Card Fail 22, 417-422.
Nagatomo, Y., McNamara, D. M., Alexis, J. D., Cooper, L. T., Dec, G. W., Pauly, D. F., Sheppard, R., Starling, R. C., Tang, W. H., and Investigators, I.-. (2017). Myocardial Recovery in Patients With Systolic Heart Failure and Autoantibodies Against beta1-Adrenergic Receptors. Journal of the American College of Cardiology 69, 968-977.
Nagatomo, Y., Yoshikawa, T., Kohno, T., Yoshizawa, A., Baba, A., Anzai, T., Meguro, T., Satoh, T., and Ogawa, S. (2009). A pilot study on the role of autoantibody targeting the beta1-adrenergic receptor in the response to beta-blocker therapy for congestive heart failure. J Card Fail 15, 224-232.
Noma, T., Lemaire, A., Naga Prasad, S. V., Barki-Harrington, L., Tilley, D. G., Chen, J., Le Corvoisier, P., Violin, J. D., Wei, H., Lefkowitz, R. J., et al. (2007). Beta-arrestin-mediated beta1-adrenergic receptor transactivation of the EGFR confers cardioprotection. J Clin Invest 117, 2445-2458.
Port, J. D., and Bristow, M. R. (2001). Altered beta-adrenergic receptor gene regulation and signaling in chronic heart failure. Journal of molecular and cellular cardiology 33, 887-905.
Prakash, A., and Markham, A. (2000). Metoprolol: a review of its use in chronic heart failure. Drugs 60, 647-678.
Rockman, H. A., Koch, W. J., and Lefkowitz, R. J. (2002). Seven-transmembrane-spanning receptors and heart function. Nature 415, 206-212.
Schur, P. H. (1988). IgG subclasses. A historical perspective. Monogr Allergy 23, 1-11.
Shenoy, S. K., Drake, M. T., Nelson, C. D., Houtz, D. A., Xiao, K., Madabushi, S., Reiter, E., Premont, R. T., Lichtarge, O., and Lefkowitz, R. J. (2006). beta-arrestin-dependent, G protein-independent ERK1/2 activation by the beta2 adrenergic receptor. J Biol Chem 281, 1261-1273.
Steinberg, S. F. (2018). Beta1-Adrenergic Receptor Regulation Revisited. Circ Res 123, 1199-1201.
Stork, S., Boivin, V., Horf, R., Hein, L., Lohse, M. J., Angermann, C. E., and Jahns, R. (2006). Stimulating autoantibodies directed against the cardiac beta1-adrenergic receptor predict increased mortality in idiopathic cardiomyopathy. Am Heart J 152, 697-704.
Vasudevan, N. T., Mohan, M. L., Goswami, S. K., and Naga Prasad, S. V. (2011a). Regulation of beta-adrenergic receptor function: an emphasis on receptor resensitization. Cell Cycle 10, 3684-3691.
Vasudevan, N. T., Mohan, M. L., Gupta, M. K., Hussain, A. K., and Naga Prasad, S. V. (2011b). Inhibition of protein phosphatase 2A activity by PI3Kgamma regulates beta-adrenergic receptor function. Mol Cell 41, 636-648.
Vasudevan, N. T., Mohan, M. L., Gupta, M. K., Martelli, E. E., Hussain, A. K., Qin, Y., Chandrasekharan, U. M., Young, D., Feldman, A. M., Sen, S., et al. (2013). Gbetagamma-independent recruitment of G-protein coupled receptor kinase 2 drives tumor necrosis factor alpha-induced cardiac beta-adrenergic receptor dysfunction. Circulation 128, 377-387.
Vidarsson, et al., (2014). IgG subclasses and allotypes: from structure to effector functions. Front Immunol 5, 520.
Wallukat, G. (2002). The beta-adrenergic receptors. Herz 27, 683-690.
Wallukat, et al., (1991). Autoantibodies against the beta-adrenergic receptor in human myocarditis and dilated cardiomyopathy: beta-adrenergic agonism without desensitization. Eur Heart J 12 Suppl D, 178-181.
Wang, et al., (2017). Galphai is required for carvedilol-induced beta1 adrenergic receptor beta-arrestin biased signaling. Nature communications 8, 1706.
Wisler et al., (2007). A unique mechanism of beta-blocker action: carvedilol stimulates beta-arrestin signaling. Proc Natl Acad Sci USA 104, 16657-16662.
Wynne, J., Braunwald, E. (1988). The cardiomyopathies and myocarditides. In Heart Disease, E. Braunwald, ed. (Philadelphia, PA: WB Saunders), pp. 1410-1457.
Youker, et al., (2014). High proportion of patients with end-stage heart failure regardless of aetiology demonstrates anti-cardiac antibody deposition in failing myocardium: humoral activation, a potential contributor of disease progression. Eur Heart J 35, 1061-1068.

Although only a few exemplary embodiments have been described in detail, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure.

What is claimed is:

1. A method of treating a subject that has cardiovascular disease (CVD), or Systemic Sclerosis (SS), comprising: treating a subject that has CVD or SS with at least one of the following:
a) IgG3 β1-Adrenergic Receptor antibodies (IgG3 β1AR antibodies) or antigen-binding portion thereof,
b) an antigenic protein that elicits the production of said IgG3 β1AR antibodies in said subject; or
c) a vector comprising a nucleic acid sequence encoding: i) said IgG3 β1AR antibodies, ii) antigen-binding portion thereof, or iii) said antigenic protein.

2. The method of claim 1, wherein said subject is negative, or lower than a control, for IgG3 β1AR antibodies prior to said treatment.

3. The method of claim 1, wherein said CVD comprises dilated cardiomyopathy.

4. The method of claim 1, wherein said IgG3 β1AR antibodies are monoclonal.

5. The method of claim 1, further comprising: testing a blood, serum, or plasma sample from said subject for the presence of IgG3 β1AR antibodies prior to said treatment.

6. The method of claim 1, further comprising: testing a blood, serum, or plasma sample from said subject for the presence of IgG3 β1AR antibodies after said treatment.

7. The method of claim 1, wherein said antigenic protein comprises at least a portion of SEQ ID NO:1.

8. The method of claim 1, wherein said antigenic protein comprises SEQ ID NO:2.

9. The method of claim 1, wherein said antigenic protein comprises at least 15 consecutive amino acids from SEQ ID NO:2.

10. The method of claim 1, wherein said antigenic protein comprises a peptide with at least 90% amino acid sequence identity with SEQ ID NO:2.

11. The method of claim 1, wherein said nucleic acid sequence comprises at least 50 consecutive nucleotides from SEQ ID NO:3.

12. The method of claim 1, wherein said subject is a human.

13. The method of claim 1, wherein said IgG3 β1AR antibodies, or antigen-binding portion thereof, are human or humanized antibodies, and said subject is a human.

14. The method of claim 1, wherein said treating comprises intravenous treatment.

15. The method of claim 1, wherein said vectors comprises a plasmid, adeno-associated virus, or adeno-associated virus.

16. The method of claim 1, wherein said subject is on a β-blocker prior to said treatment.

17. The method of claim 1, further comprising: administering a β-blocker to said subject prior to, during, or after said treatment.

18. The method of claim 17, wherein said β-blocker is selected from the group consisting of: Acebutolol, Atenolol, Bisoprolol, Metoprolol, Nadolol, Nebivolol, and Propranolol.

19. The method of claim 1, wherein said IgG3 β1AR antibodies are autoantibodies purified from a blood, serum, or plasma sample of a donor or the subject themselves.

20. A kits, system, or composition comprising:
a) at least one of the following:
i) IgG3 β1-Adrenergic Receptor antibodies (IgG3 β1AR antibodies) or antigen-binding portion thereof;
ii) an antigenic protein that elicits the production of IgG3 β1AR antibodies in a subject; or
iii) a vector comprising a nucleic acid sequence encoding: i) said IgG3 β1AR antibodies, ii) said antigen-binding portion thereof, or iii) said antigenic protein, and
b) a β-blocker.

21. A method comprising:
a) receiving results of, or conducting, an IgG3 β1-Adrenergic Receptor IgG3 β1AR antibodies (IgG3 β1AR antibodies) level analysis on a sample from a subject with cardiovascular disease (CVD) or Systemic Sclerosis (SS), and
b) performing at least one of the following after identifying said sample as having higher levels of said IgG3 β1AR antibodies compared to control levels,
i) treating said subject with a beta-blocker;
ii) treating said subject with IgG3 β1AR antibodies or antigen-binding portion thereof;
iii) treating said subject with an antigenic protein that elicits the production of said IgG3 β1AR antibodies in said subject;
iv) a vector comprising a nucleic acid sequence encoding: i) said IgG3 β1AR antibodies, ii) said antigen-binding portion thereof, or iii) said antigenic protein; and/or
v) providing a report to said patient or medical personnel treating said patient, indicating said subject is suitable for, or should be, treated with: A) said beta-blocker, B) said IgG3 β1AR antibodies or antigen-binding portion thereof, C) said antigenic protein, and/or D) said vector.

22. The method of claim 21, wherein said control levels are from the general population or CVD patients not stratified by IgG3 β1AR antibody levels.
